SEQUENCE LISTING (2 sequences)

SEQ ID NO: 1 - protein, 6 aa, Artificial Sequence
  Synthetic peptide
CDDRDE

SEQ ID NO: 2 - protein, 5 aa, Artificial Sequence
  Synthetic peptide
DRDDC

(19) United States
(12) Patent Application Publication
(10) Pub. No.: US 2013/0266964 A1
(43) Pub. Date: Oct. 10, 2013

(54) METHOD OF DETECTING ENDOTHELIAL PROGENITOR CELLS

(71) Applicant: PURDUE RESEARCH FOUNDATION, West Lafayette, IN (US)

(72) Inventors: Philip Stewart LOW, West Lafayette, IN (US); Andrew Richard HILGENBRINK, Dallas, TX (US)

(21) Appl. No.: 13/910,306

(22) Filed: Jun. 5, 2013

Related U.S. Application Data
(62) Division of application No. 12/301,864, filed on Nov. 21, 2008, now abandoned.
(60) Provisional application No. 60/802,648, filed on May 23, 2006.

(51) Int. Cl. G01N 33/50

(52) U.S. Cl. CPC G01N 33/5005
USPC 435/7.21

(57) ABSTRACT

A method of detecting endothelial progenitor cells is provided. The method involves contacting a population of cells with a composition comprising a conjugate or complex of the formula A_{b}-X where the group A_{b} comprises a folate that binds to endothelial progenitor cells, and the group X comprises a fluorescent chromophore, and eliciting a fluorescent response from bound A_{b}-X.

### CROSS REFERENCE TO RELATED APPLICATIONS

This application is a divisional of U.S. application Ser. No. 12/301,864 filed on Nov. 21, 2008, which is a U.S. national stage entry under 35 U.S.C. §371 of international application no. PCT/US07/012,269, filed May 23, 2007, which claims priority under 35 U.S.C. §119(e) to U.S. provisional application No. 60/802,648, filed May 23, 2006, the contents of each of which are incorporated herein by reference in their entirety.

### FIELD OF THE INVENTION

This invention relates to methods for treating and diagnosing disease states worsened by progenitor cells. More particularly, ligands that bind to progenitor cells are complexed with a quantifiable marker for use in diagnosis or to an antigen, a cytotoxin, or an agent for altering progenitor cell function for use in the treatment of disease states worsened by progenitor cells.

### BACKGROUND

The mammalian immune system provides a means for the recognition and elimination of foreign pathogens. While the immune system normally provides a line of defense against foreign pathogens, there are many instances where the immune response itself is involved in the progression of disease. Exemplary of diseases caused or worsened by the host's own immune response are autoimmune diseases and other diseases in which the immune response contributes to pathogenesis. For example, macrophages are generally the first cells to encounter foreign pathogens, and accordingly, they play an important role in the immune response, but activated macrophages can also contribute to the pathophysiology of disease in some instances.

The folate receptor is a 38 KD GPI-anchored protein that binds the vitamin folic acid with high affinity (<1 nM). Following receptor binding, rapid endocytosis delivers a substantial fraction of the vitamins into the cell, where they are unloaded in an endosomal compartment at low pH. Importantly, covalent conjugation of small molecules, proteins, and even liposomes to folic acid does not block the vitamin's ability to bind the folate receptor, and therefore, folate-drug conjugates can readily be delivered to and can enter cells by receptor-mediated endocytosis.

Because most cells use an unrelated reduced folate carrier to acquire the necessary folic acid, expression of the folate receptor is restricted to a few cell types. With the exception of kidney, choroid plexus, and placenta, normal tissues express low or nondetectable levels of the folate receptor. However, many malignant tissues, including ovarian, breast, bronchial, and brain cancers express significantly elevated levels of the receptor. In fact, it is estimated that 95% of all ovarian carcinomas overexpress the folate receptor. It has been reported that the folate receptor β, the nonepithelial isoform of the folate receptor, is expressed on activated (but not resting) synovial macrophages. Thus, folate receptors are expressed on a subset of macrophages (i.e., activated macrophages).

### SUMMARY

It is unknown, however, whether folate receptors are expressed on progenitor cells such as CD133⁺ Flk1⁺ cells commonly referred to as endothelial progenitor cells, or on common progenitor cells for both endothelial progenitor cells and macrophages. Thus, Applicants have undertaken to determine whether folate receptors are expressed on these progenitor cells and whether progenitor cell targeting, using a ligand such as folate, to deliver cytotoxic or other inhibitory compounds to these cells, is useful therapeutically. Applicants have also undertaken to determine whether a quantifiable marker linked to a ligand capable of binding to progenitor cells, such as CD133⁺ Flk1⁺ endothelial progenitor cells or common precursor cells for both endothelial progenitor cells and macrophages, may be useful for diagnosing inflammatory pathologies, and other pathologies that involve vasculogenesis.

A method is provided for treating and diagnosing disease states worsened by progenitor cells. In one embodiment, the progenitor cells are CD133⁺ Flk1⁺ endothelial progenitor cells. In another embodiment, the CD133⁺ Flk1⁺ cells are activated progenitor cells. In one embodiment, disease states worsened by CD133⁺ Flk1⁺ endothelial progenitor cells are treated by delivering an antigen to the cells, by linking the antigen to a ligand that binds to these cells, to redirect host immune responses to CD133⁺ Flk1⁺ endothelial progenitor cells. In another embodiment, CD133⁺ Flk1⁺ endothelial progenitor cells can be inactivated or killed by other methods such as by the delivery to these cells of cytotoxins or other compounds capable of altering their function. In similar embodiments, the progenitor cells can be common progenitor cells for both endothelial progenitor cells and macrophages.

In one embodiment, an antigen is delivered to CD133⁺ Flk1⁺ endothelial progenitor cells to inactivate or kill these cells. In this embodiment, ligands that bind to CD133⁺ Flk1⁺ endothelial progenitor cells can be conjugated with an antigen to redirect host immune responses to the these cells, or the ligand can be conjugated to a cytotoxin for killing of these cells. Ligands that can be used in the conjugates of the present invention include those that bind to receptors expressed on CD133⁺ Flk1⁺ endothelial progenitor cells, such as the folate receptor, or ligands such as monoclonal antibodies directed to cell surface markers expressed on CD133⁺ Flk1⁺ endothelial progenitor cells or other ligands that bind to these cells. In another embodiment, ligands that bind to CD133⁺ Flk1⁺ endothelial progenitor cells are conjugated to a quantifiable marker and the conjugate is used to diagnose diseases worsened by CD133⁺ Flk1⁺ endothelial progenitor cells. In similar embodiments, the progenitor cells can be common progenitor cells for both endothelial progenitor cells and macrophages. In this embodiment, ligands that bind to the common precursor cells can be used.

In another embodiment, a method is provided for diagnosing a disease state worsened by CD133⁺ Flk1⁺ endothelial progenitor cells. The method comprises the steps of isolating CD133⁺ Flk1⁺ endothelial progenitor cells from a patient suffering from a disease state worsened by CD133⁺ Flk1⁺ endothelial progenitor cells, contacting the endothelial progenitor cells with a composition comprising a conjugate or complex of the general formula

A_{b}-X

where the group A_{b} comprises a vitamin, or an analog thereof, that binds to the progenitor cells and the group X comprises a quantifiable marker, and quantifying the percentage of CD133⁺ Flk1⁺ endothelial progenitor cells that expresses a receptor for the vitamin. In another embodiment, A_{b} comprises folate, or an analog thereof. In yet another embodiment, A_{b} comprises a CD133⁺ Flk1⁺ endothelial progenitor cell-binding antibody or antibody fragment or other ligands that bind to CD133⁺ Flk1⁺ endothelial progenitor cells. In another embodiment, the quantifiable marker comprises a metal chelating moiety that binds an element that is a radionuclide. In still another embodiment, the quantifiable marker comprises a chromophore selected from the group consisting of fluorescein, Oregon Green, rhodamine, phycoerythrin, Texas Red, and AlexaFluor 488, or another appropriate fluorescent chromophore. In similar embodiments, the progenitor cells can be common progenitor cells for both endothelial progenitor cells and macrophages. In this embodiment, ligands that bind to the common precursor cells can be used.

In another embodiment, a method is provided for diagnosing a disease state worsened by CD133⁺ Flk1⁺ endothelial progenitor cells. The method comprises the steps of administering parenterally to a patient a composition comprising a conjugate or complex of the general formula

A_{b}-X

where the group A_{b} comprises a vitamin, or an analog thereof, that binds to CD133⁺ Flk1⁺ endothelial progenitor cells and the group X comprises a quantifiable marker, and quantifying the percentage of CD133⁺ Flk1⁺ endothelial progenitor cells that expresses a receptor for the vitamin. In similar embodiments, the progenitor cells can be common progenitor cells for both endothelial progenitor cells and macrophages. The quantifiable marker can be, for example, a radioactive probe, a fluorescent probe, an enzyme capable of amplifying a signal, an antibody capable of assisting in amplifying a signal, or other agents for use in amplifying a signal, such as oligonucleotides.

In another embodiment, a method is provided for treating a disease state worsened by CD133⁺ Flk1⁺ endothelial progenitor cells. The method comprises the steps of administering to a patient suffering from a disease state worsened by CD133⁺ Flk1⁺ endothelial progenitor cells an effective amount of a composition comprising a conjugate or complex of the general formula

A_{b}-X

where the group A_{b} comprises a vitamin, or an analog thereof, that binds to CD133⁺ Flk1⁺ endothelial progenitor cells and the group X comprises an antigen, a cytotoxin, or a compound capable of altering the function of the progenitor cells, and eliminating the disease state. In similar embodiments, the progenitor cells can be common progenitor cells for both endothelial progenitor cells and macrophages.

In yet another embodiment, a compound for diagnosing or treating a disease state worsened by progenitor cells, such as CD133⁺ Flk1⁺ endothelial progenitor cells or common progenitor cells for both endothelial progenitor cells and macrophages is provided. The compound is selected from the following group of compounds:

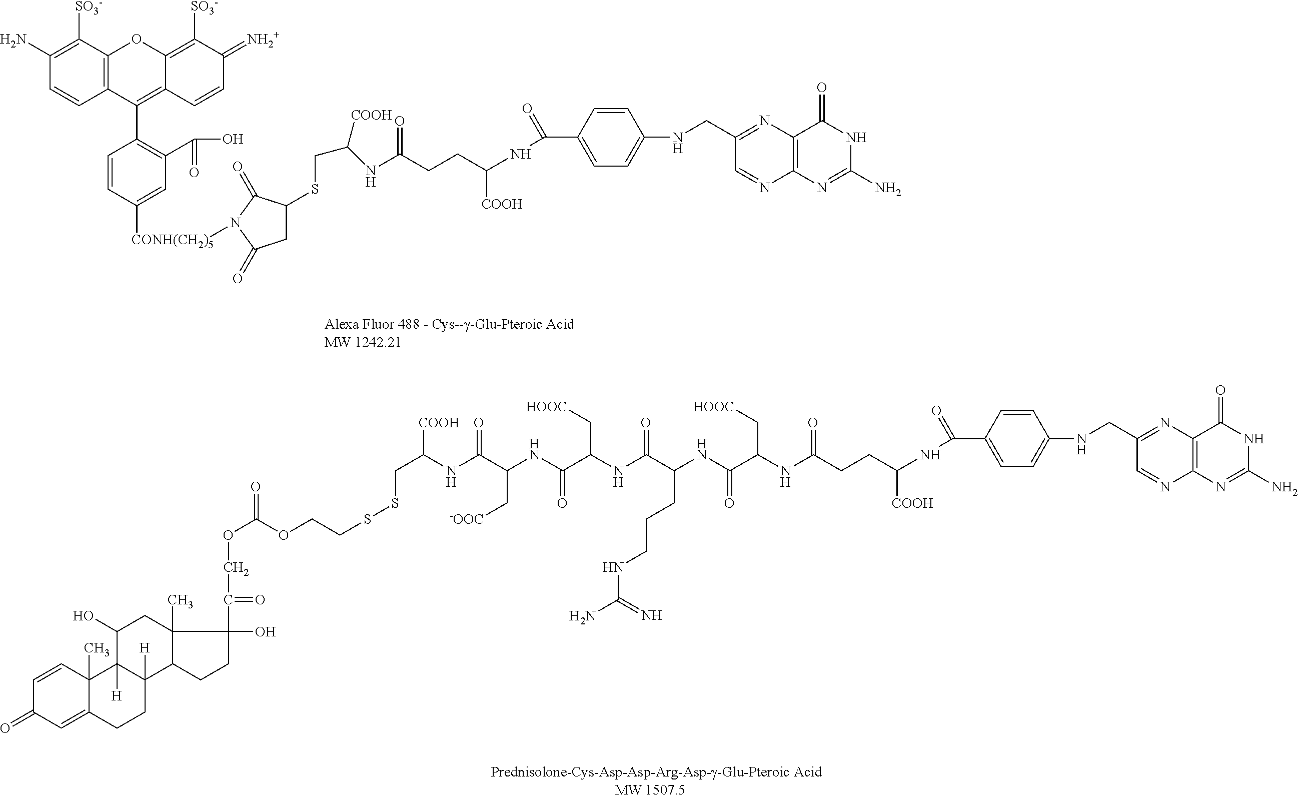

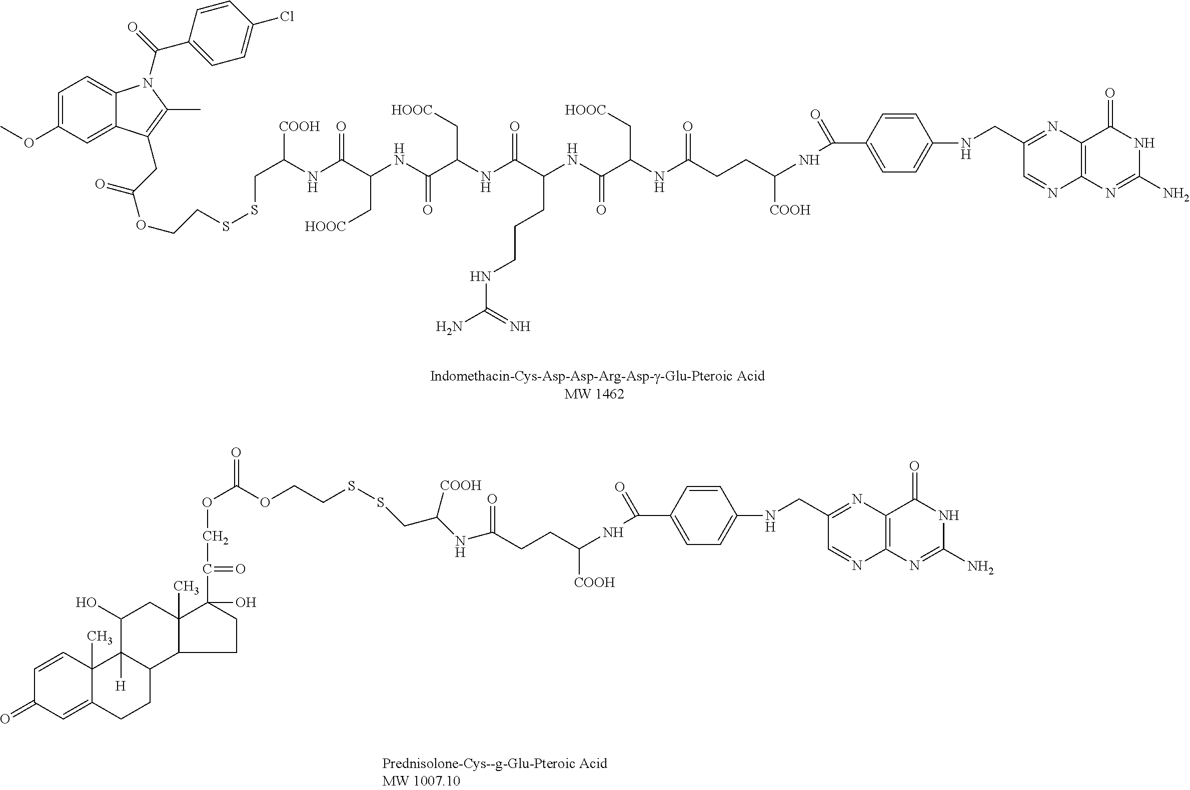

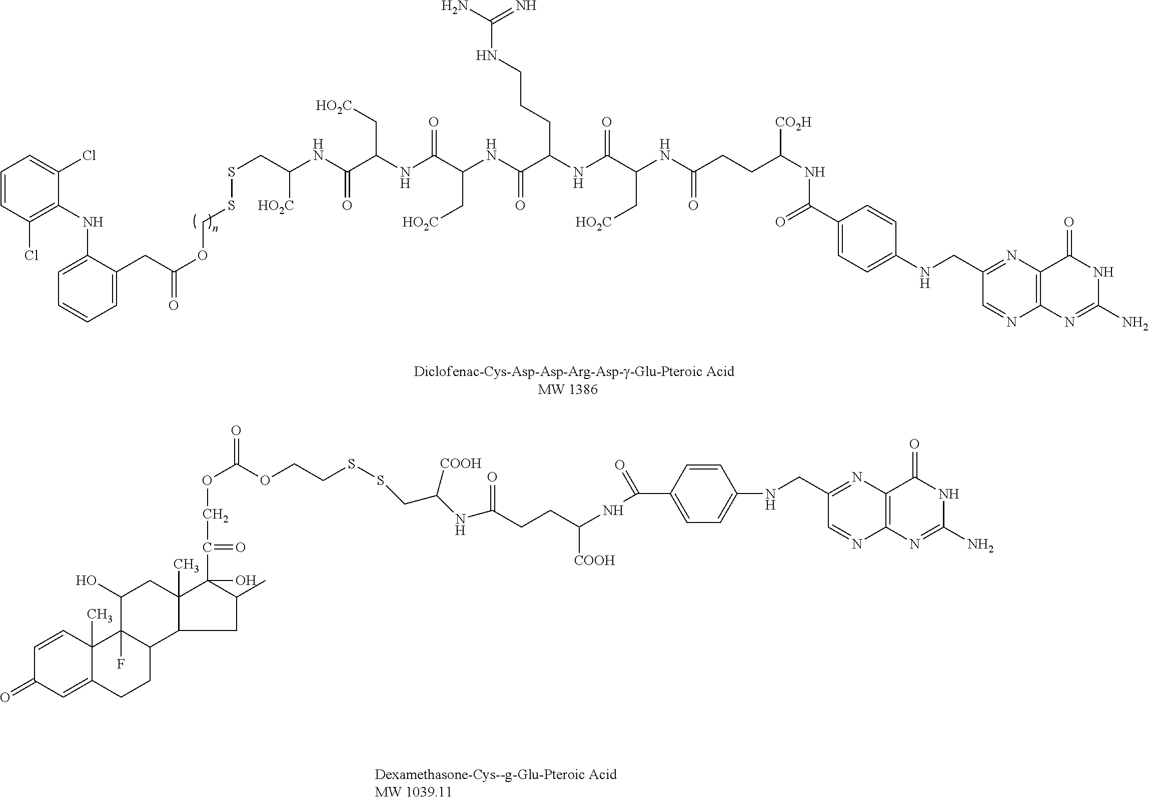

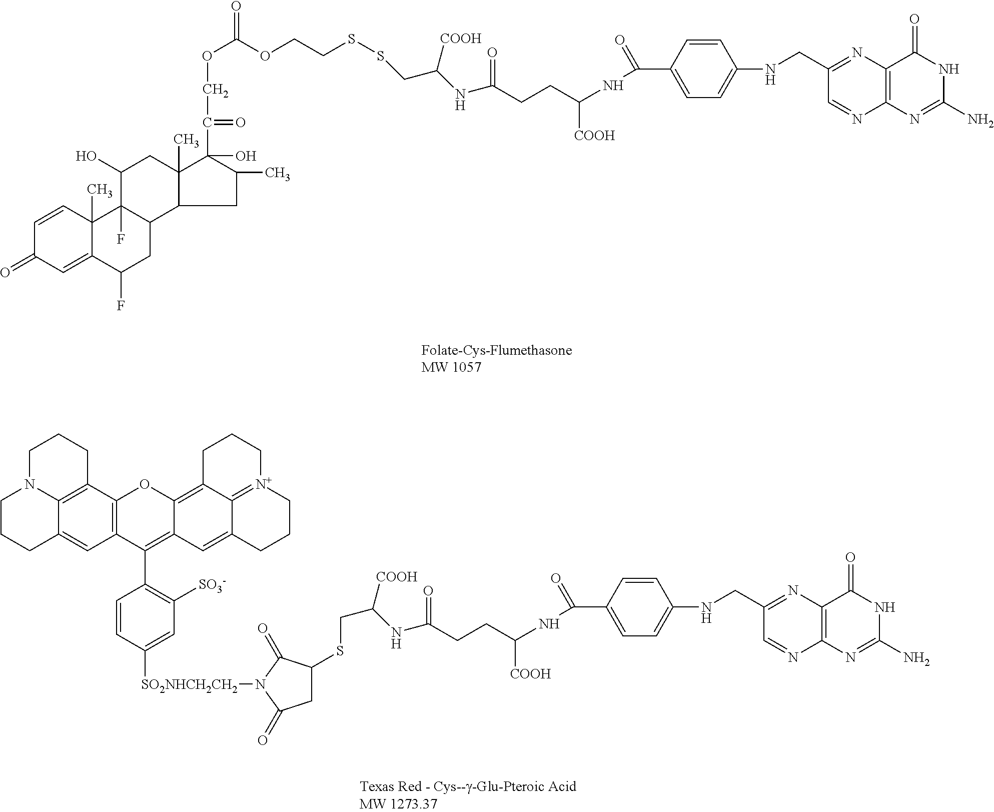

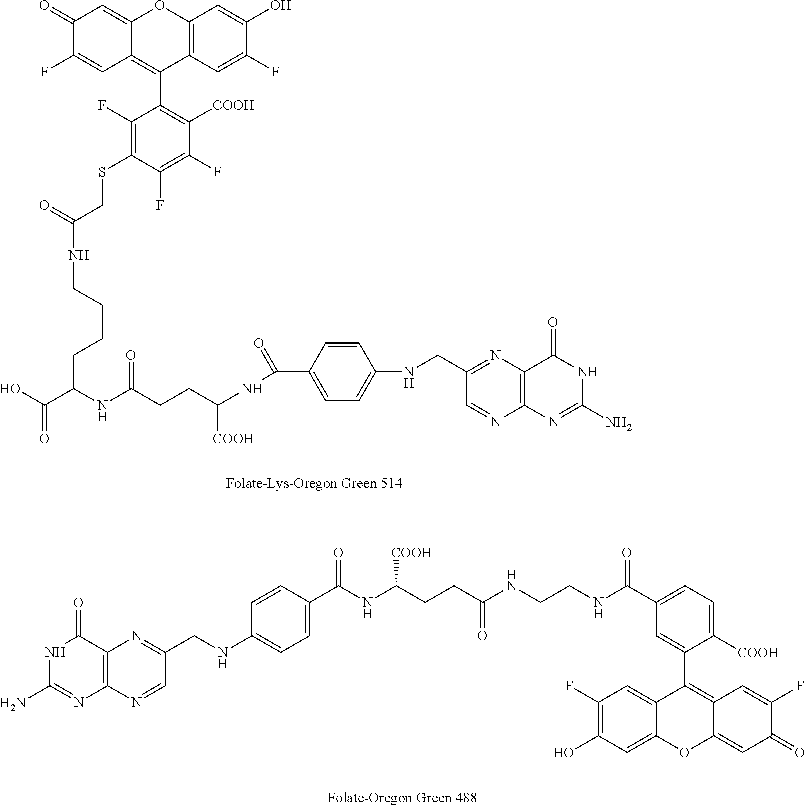 [SMILES: Nc1nc2ncc(CNc3ccc(C(=O)NC(CCC(=O)NC(CCCCNC(=O)CSc4c(F)c(F)c(C(=O)O)c(-c5c6cc(F)c(=O)cc-6oc6cc(O)c(F)cc56)c4F)C(=O)O)C(=O)O)cc3)nc2c(=O)[nH]1 ; Nc1nc2ncc(CNc3ccc(C(=O)N[C@@H](CCC(=O)NCCNC(=O)c4ccc(C(=O)O)c(-c5c6cc(F)c(=O)cc-6oc6cc(O)c(F)cc56)c4)C(=O)O)cc3)nc2c(=O)[nH]1]

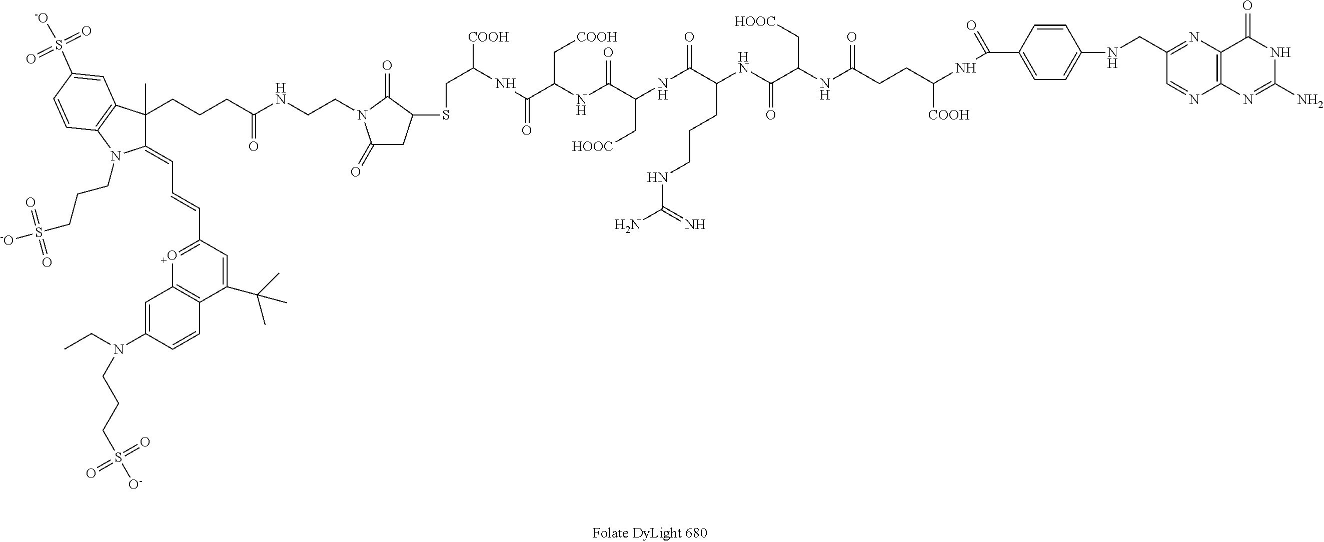

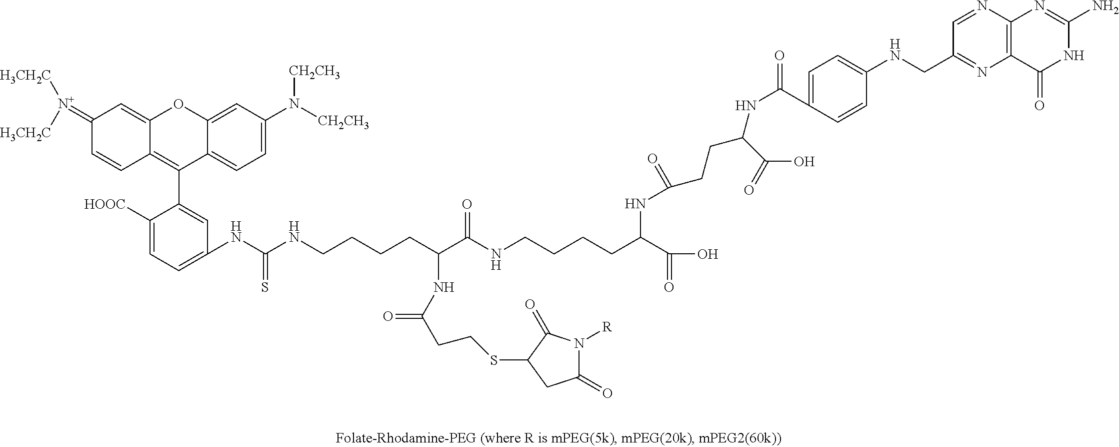

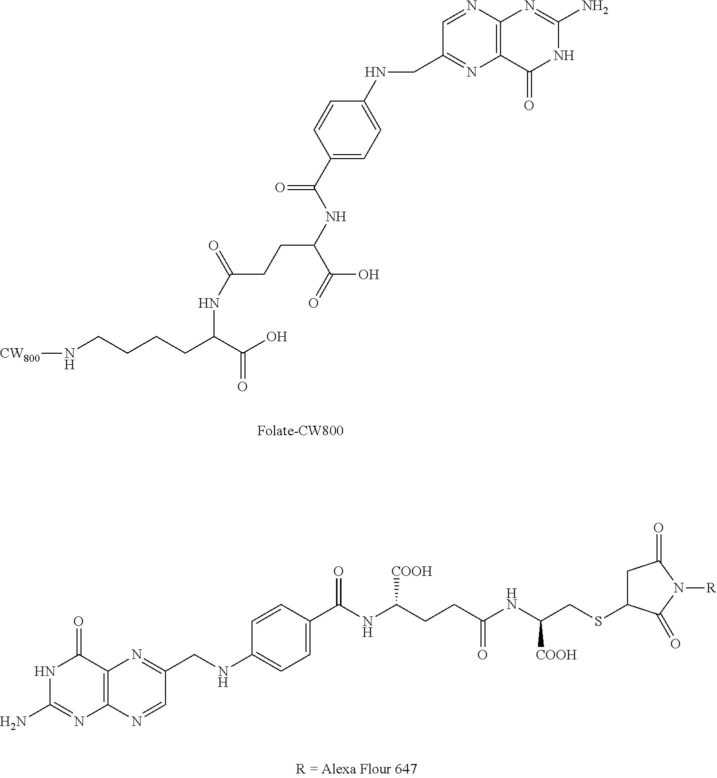

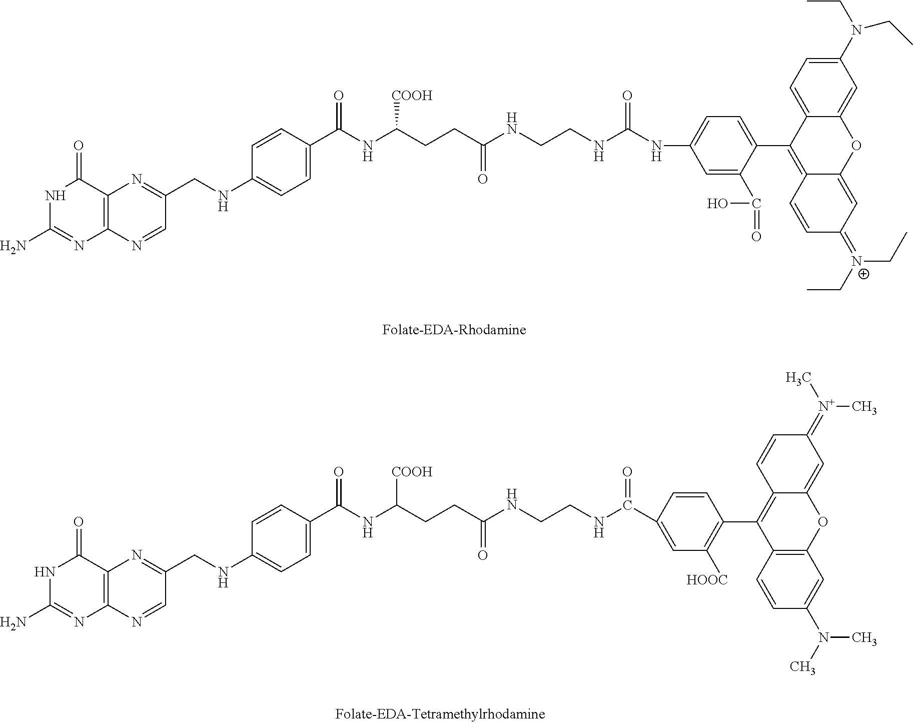 [SMILES: CCN(CC)c1ccc2c(-c3ccc(NC(=O)NCCNC(=O)CC[C@H](NC(=O)c4ccc(NCc5cnc6nc(N)[nH]c(=O)c6n5)cc4)C(=O)O)cc3C(=O)O)c3ccc(=[N+](CC)CC)cc-3oc2c1 ; CN(C)c1ccc2c(-c3ccc(C(=O)NCCNC(=O)CCC(NC(=O)c4ccc(NCc5cnc6nc(N)[nH]c(=O)c6n5)cc4)C(=O)O)cc3C(=O)O)c3ccc(=[N+](C)C)cc-3oc2c1]

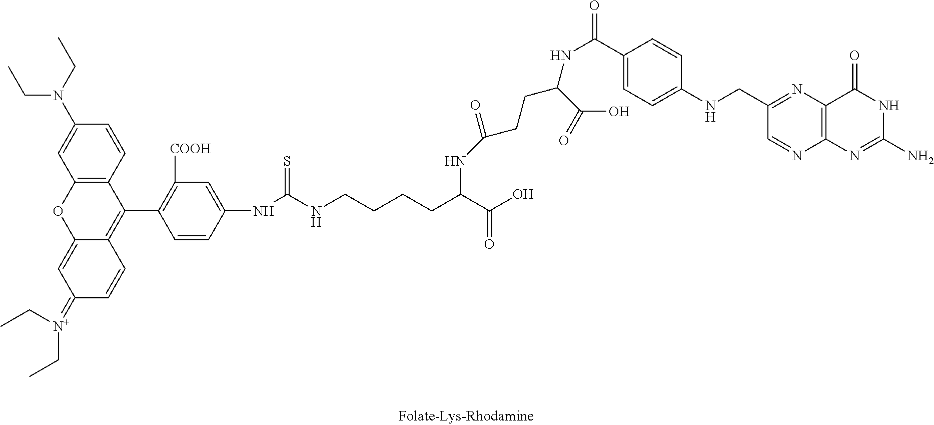 [SMILES: CCN(CC)c1ccc2c(-c3ccc(NC(=S)NCCCCC(NC(=O)CCC(NC(=O)c4ccc(NCc5cnc6nc(N)[nH]c(=O)c6n5)cc4)C(=O)O)C(=O)O)cc3C(=O)O)c3ccc(=[N+](CC)CC)cc-3oc2c1]

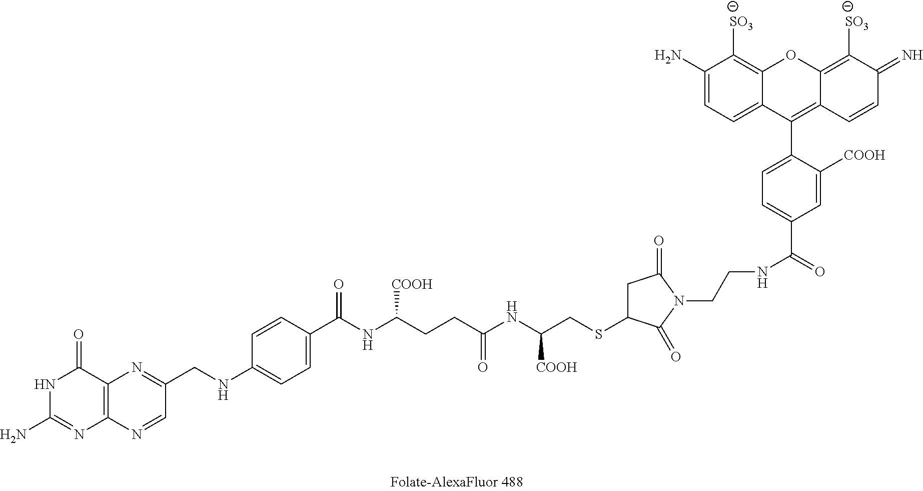

In another embodiment, a method of quantifying endothelial progenitor cells is provided. The method comprises the steps of isolating the progenitor cells from a patient suffering from a disease state mediated by the progenitor cells, contacting the progenitor cells with a composition comprising a conjugate or complex of the general formula A_{b}-X where the group A_{b} comprises a vitamin, or an analog thereof, that binds to endothelial progenitor cells and the group X comprises a quantifiable marker, and quantifying the percentage of progenitor cells that expresses a receptor for the vitamin.

In another embodiment, a use is provided of a composition comprising a conjugate or complex of the general formula A_{b}-X where the group A_{b} comprises a vitamin, or an analog thereof, that binds to the progenitor cells and the group X comprises an antigen, a cytotoxin, or a compound capable of altering progenitor cell function in the manufacture of a medicament for use in treating a disease state worsened by progenitor cells.

In yet another embodiment, a method of quantifying endothelial progenitor cells is provided. The method comprises the steps of contacting the progenitor cells in a patient suffering from a disease state mediated by the progenitor cells with a composition comprising a conjugate or complex of the general formula A_{b}-X where the group A_{b} comprises a vitamin, or an analog thereof, that binds to endothelial progenitor cells and the group X comprises a quantifiable marker, and quantifying the percentage of progenitor cells that expresses a receptor for the vitamin.

### BRIEF DESCRIPTION OF THE DRAWINGS

FIG. 1 shows flow cytometry analysis, using Flk1 (A), CD115 (B), CD69 (C), CD11b (D), CD8a (E), and CD25 (F) antibodies and folate-FITC, of markers that are co-expressed with the folate receptor on CD133⁺ Flk1⁺ endothelial progenitor cells or on common precursor cells to both endothelial progenitor cells and macrophages.

FIG. 2 shows flow cytometry analysis, using CD62L (A), CD80 (B), CD86 (C), CD44 (D), CD23 (E), and CD14 (F) antibodies and folate-FITC, of markers that are co-expressed with the folate receptor on CD133⁺ Flk1⁺ endothelial progenitor cells.

FIG. 3 shows flow cytometry analysis, using Ly-6 (A), F4/80 (B), CD49d (C), CD16.2/32.2 (D), and MHC Class II (E) antibodies and folate-FITC, of markers that are co-expressed with the folate receptor on CD133⁺ Flk1⁺ endothelial progenitor cells or on common precursor cells to both endothelial progenitor cells and macrophages.

FIG. 4 shows folate-fluorescein (folate-FITC) binding, quantified by flow cytometry, to CD133⁺ endothelial progenitor cells (panel A), to Flk-1⁺ endothelial progenitor cells (Panel B), and to CD44⁺ endothelial progenitor cells (panel C) without excess unlabeled folic acid (top panels) or preincubated with an excess of unlabeled folic acid (bottom panels (Competed Samples)) to compete with folate-FITC for binding.

FIG. 5 shows folate-fluorescein (folate-FITC) binding, quantified by flow cytometry, to Ly-6⁺ endothelial progenitor cells (panel A), to CD25⁺ endothelial progenitor cells (Panel B), and to CD62-L⁺ endothelial progenitor cells (panel C) without excess unlabeled folic acid (top panels) or preincubated with an excess of unlabeled folic acid (bottom panels (Competed Samples)) to compete with folate-FITC for binding.

### DETAILED DESCRIPTION

Methods are provided for treating and diagnosing disease states worsened (e.g., caused or augmented) by progenitor cells, such as CD133⁺ Flk-1⁺ endothelial progenitor cells or common precursor cells to both endothelial progenitor cells and macrophages (i.e., referred to in this application as “common precursor cells”). Exemplary disease states include fibromyalgia, rheumatoid arthritis, osteoarthritis, ulcerative colitis, Crohn's disease, psoriasis, osteomyelitis, multiple sclerosis, atherosclerosis, pulmonary fibrosis, sarcoidosis, systemic sclerosis, organ transplant rejection (GVHD), lupus erythematosus, Sjögren's syndrome, glomerulonephritis, inflammations of the skin (e.g., psoriasis), cancer, proliferative retinopathy, restenosis, and chronic inflammations. Such disease states can be diagnosed by isolating the progenitor cells from a patient suffering from such disease state, contacting the cells with a composition comprising a conjugate of the general formula A_{b}-X wherein the group A_{b} comprises a ligand that binds to the progenitor cells, and the group X comprises a quantifiable marker, and quantifying the percentage of the progenitor cells expressing a receptor for the ligand.

As used herein, the phrase “progenitor cells” includes CD133⁺ and/or Flk1⁺ endothelial progenitor cells and common precursor cells for both endothelial progenitor cells and macrophages.

As used herein, the phrase “common precursor cells” refers to common precursor cells for both endothelial progenitor cells and macrophages. These cells have CD133 and/or Flk1 markers and also have a marker selected from the group consisting of CD11b, F4/80, and CD115.

As used herein, the terms “eliminated” and “eliminating” in reference to the disease state, mean reducing the symptoms or eliminating the symptoms of the disease state or preventing the progression or the reoccurrence of disease.

As used herein, the terms “elimination” and “deactivation” of the progenitor cell population that expresses the ligand receptor mean that this progenitor cell population is killed or is completely or partially inactivated which reduces the pathogenesis characteristic of the disease state being treated.

As used herein, “worsened by” in reference to diseases worsened by progenitor cells means caused by or augmented by. For example, endothelial progenitor cells can directly cause disease or can augment disease states such as by stimulating other immune cells to secrete factors that worsen disease states, such as by stimulating T-cells to secrete TNF-α, or by increasing the blood supply (e.g., by vasculogenesis) to pathologic tissues, such as cancer tissues. Illustratively, endothelial progenitor cells themselves may also harbor infections and cause disease and infected progenitor cells may cause other immune cells to secrete factors that cause disease such as TNF-α secretion by T-cells.

Such disease states can also be diagnosed by administering parenterally to a patient a composition comprising a conjugate or complex of the general formula A_{b}-X where the group A_{b} comprises a ligand that binds to progenitor cells and the group X comprises a quantifiable marker, and quantifying the percentage of the cells that expresses a receptor for the ligand.

CD133⁺ Flk-1⁺ endothelial progenitor cell-worsened disease states can be treated in accordance with the methods disclosed herein by administering an effective amount of a composition A_{b}-X wherein A_{b} comprises a ligand that binds to CD133⁺ Flk-1⁺ endothelial progenitor cells and wherein the group X comprises an antigen, a cytotoxin, or a compound capable of altering the function of the endothelial progenitor cells. Such targeting conjugates, when administered to a patient suffering from a disease state augmented by the endothelial progenitor cells, work to concentrate and associate the conjugated cytotoxin, antigen, or compound capable of altering endothelial progenitor cell function with the population of endothelial progenitor cells to kill the cells or alter cell function. The conjugate is typically administered parenterally, but can be delivered by any suitable method of administration (e.g., orally), as a composition comprising the conjugate and a pharmaceutically acceptable carrier therefor. Conjugate administration is typically continued until symptoms of the disease state are reduced or eliminated, or administration is continued after this time to prevent progression or reappearance of the disease. The cells may be common precursor cells in similar embodiments and in these embodiments ligands that bind to common precursor cells can be used.

In one embodiment, disease states worsened by progenitor cells are diagnosed in a patient by isolating the cells from the patient, contacting the progenitor cells with a conjugate A_{b}-X wherein A_{b} comprises a ligand that binds to the progenitor cells and X comprises a quantifiable marker, and quantifying the percentage of progenitor cells expressing the receptor for the ligand. In another embodiment, the diagnostic conjugates can be administered to the patient as a diagnostic composition comprising a conjugate and a pharmaceutically acceptable carrier and thereafter the progenitor cells can be collected from the patient to quantify the percentage of cells expressing the receptor for the ligand A_{b}. In this embodiment, the composition is typically formulated for parenteral administration and is administered to the patient in an amount effective to enable quantification of the progenitor cells. In another embodiment, disease states can also be diagnosed by administering parenterally to a patient a composition comprising a conjugate or complex of the general formula A_{b}-X where the group A_{b} comprises a ligand that binds to progenitor cells and the group X comprises a quantifiable marker, and quantifying the percentage of the cells that expresses a receptor for the ligand.

In one embodiment, for example, the quantifiable marker (e.g., a reporter molecule) can comprise a radiolabeled compound such as a chelating moiety and an element that is a radionuclide, for example a metal cation that is a radionuclide. In another embodiment, the radionuclide is selected from the group consisting of technetium, gallium, indium, and a positron emitting radionuclide (PET imaging agent). In another embodiment, the quantifiable marker can comprise a fluorescent chromophore such as, for example, fluorescein, rhodamine, Texas Red, phycoerythrin, Oregon Green, AlexaFluor 488 (Molecular Probes, Eugene, Oreg.), Cy3, Cy5, Cy7, and the like.

Diagnosis typically occurs before treatment. However, in the diagnostic methods described herein, the term “diagnosis” can also mean monitoring of the disease state before, during, or after treatment to determine the progression of the disease state. The monitoring can occur before, during, or after treatment, or combinations thereof, to determine the efficacy of therapy, or to predict future episodes of disease. The quantification can be performed by any suitable method known in the art, including imaging methods, such as intravital imaging.

The method disclosed herein can be used for both human clinical medicine and veterinary applications. Thus, the host animal afflicted with the disease state worsened by progenitor cells and in need of diagnosis or therapy can be a human, or in the case of veterinary applications, can be a laboratory, agricultural, domestic or wild animal. In embodiments where the conjugates are administered to the patient or animal, the conjugates can be administered parenterally to the animal or patient suffering from the disease state, for example, intradermally, subcutaneously, intramuscularly, intraperitoneally, or intravenously. Alternatively, the conjugates can be administered to the animal or patient by other medically useful procedures and effective doses can be administered in standard or prolonged release dosage forms, such as a slow pump. The therapeutic method described herein can be used alone or in combination with other therapeutic methods recognized for the treatment of inflammatory disease states, or disease states augmented by vasculogenesis.

In the ligand conjugates of the general formula A_{b}-X, the group A_{b} is a ligand that binds to CD133⁺ Flk-1⁺ endothelial progenitor cells or common precursor cells when the conjugates are used to diagnose or treat disease states. Any of a wide number of binding ligands can be employed. Acceptable ligands include particularly folate receptor binding ligands, and analogs thereof, and antibodies or antibody fragments capable of recognizing and binding to surface moieties expressed or presented on CD133⁺ Flk-1⁺ endothelial progenitor cells or on common precursor cells. Antagonists and agonists for CD133, Flk1, CD11b, F4/80, or CD115 may be acceptable ligands. In one embodiment, the binding ligand is folic acid, a folic acid analog, or another folate receptor binding molecule. In another embodiment the binding ligand is a specific monoclonal or polyclonal antibody or an Fab or an scFv (i.e., a single chain variable region) fragment of an antibody capable of binding to CD133⁺ Flk-1⁺ endothelial progenitor cells or to common precursor cells.

In one embodiment, the binding ligand can be folic acid, a folic acid analog, or another folate receptor-binding molecule. Analogs of folate that can be used include folinic acid, pteropolyglutamic acid, and folate receptor-binding pteridines such as tetrahydropterins, dihydrofolates, tetrahydrofolates, and their deaza and dideaza analogs. The terms “deaza” and “dideaza” analogs refers to the art recognized analogs having a carbon atom substituted for one or two nitrogen atoms in the naturally occurring folic acid structure. For example, the deaza analogs include the 1-deaza, 3-deaza, 5-deaza, 8-deaza, and 10-deaza analogs. The dideaza analogs include, for example, 1,5 dideaza, 5,10-dideaza, 8,10-dideaza, and 5,8-dideaza analogs. The foregoing folic acid analogs are conventionally termed “folates,” reflecting their capacity to bind to folate receptors. Other folate receptor-binding analogs include aminopterin, amethopterin (methotrexate), N¹⁰-methylfolate, 2-deamino-hydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate).

In another embodiment, other vitamins can be used as the binding ligand. The vitamins that can be used in accordance with the methods described herein include niacin, pantothenic acid, folic acid, riboflavin, thiamine, biotin, vitamin B₁₂, vitamins A, D, E and K, other related vitamin molecules, analogs and derivatives thereof, and combinations thereof.

In other embodiments, the binding ligand can be any ligand that binds to a receptor expressed or overexpressed on endothelial progenitor cells or common precursor cells including CD133, Flk1, CD11b, CD115, CD69, CD8a, CD25, CD62L, CD80, CD86, CD44, CD23, CD14, Ly-6, F4/80, CD49d, CD16.2/32.2, and the like. Examples of such ligands include both antagonists and agonists for each of the above membrane-spanning proteins.

The targeted conjugates used for diagnosing or treating disease states mediated by progenitor cells have the formula A_{b}-X, wherein A_{b} is a ligand capable of binding to the progenitor cells, and the group X comprises a quantifiable marker or an antigen (such as an immunogen), cytotoxin, or a compound capable of altering progenitor cell function. In such conjugates wherein the group A_{b} is folic acid, a folic acid analog, or another folic acid receptor binding ligand, these conjugates are described in detail in U.S. Pat. No. 5,688,488, the specification of which is incorporated herein by reference. That patent, as well as related U.S. Pat. Nos. 5,416,016 and 5,108,921, and related U.S. Patent Publication Serial No. US 2005/0002942 A1, each incorporated herein by reference, describe methods and examples for preparing conjugates useful in accordance with the methods described herein. The present targeted diagnostic and therapeutic agents can be prepared and used following general protocols described in those earlier patents and patent applications, and by the protocols described herein.

In accordance with another embodiment, there is provided a method of treating disease states worsened by progenitor cells by administering to a patient suffering from such disease state an effective amount of a composition comprising a conjugate of the general formula A_{b}-X wherein A_{b} is as defined above and the group X comprises a cytotoxin, an antigen (i.e., a compound that elicits an immune response in vivo), or a compound capable of altering progenitor cell function. In these embodiments, the progenitor cells can be activated cells and the group A_{b} can be any of the ligands described above. Exemplary of cytotoxic moieties useful for forming conjugates for use in accordance with the methods described herein are clodronate, anthrax, Pseudomonas exotoxin, typically modified so that these cytotoxic moieties do not bind to normal cells, and other toxins or cytotoxic agents including art-recognized chemotherapeutic agents such as adrenocorticoids, alkylating agents, antiandrogens, antiestrogens, androgens, estrogens, antimetabolites such as cytosine arabinoside, purine analogs, pyrimidine analogs, and methotrexate, busulfan, carboplatin, chlorambucil, cisplatin and other platinum compounds, tamoxiphen, taxol, cyclophosphamide, plant alkaloids, prednisone, hydroxyurea, teniposide, and bleomycin, nitrogen mustards, nitrosureas, vincristine, vinblastine, MEK kinase inhibitors, MAP kinase pathway inhibitors, PI-3-kinase inhibitors, mitochondrial perturbants, NFκB pathway inhibitors, proteosome inhibitors, pro-apoptotic agents, glucocorticoids, such as prednisolone, flumethasone, dexamethasone, and betamethasone, indomethacin, diclofenac, non-steroidal anti-inflammatory agents, cyclooxygenase inhibitors, lipooxygenase inhibitors, apoptosis-inducing agents, proteins such as pokeweed, saporin, momordin, and gelonin, non-steroidal anti-inflammatory drugs (NSAIDs), protein synthesis inhibitors, didemnin B, verrucarin A, geldanamycin, and the like. Such toxins or cytotoxic compounds can be directly conjugated to the targeting ligand, for example, folate or another folate receptor-binding ligand, or they can be formulated in liposomes or other small particles which themselves are targeted as conjugates of the progenitor cell-binding ligand typically by covalent linkages to component phospholipids.

Similarly, when the group X comprises a compound capable of altering progenitor cell function, for example, a cytokine such as IL-10 or IL-11, the compound can be covalently linked to the targeting ligand A_{b}, for example, a folate receptor-binding ligand or a progenitor cell-binding antibody or antibody fragment directly, or the function altering compound can be encapsulated in a liposome which is itself targeted to progenitor cells by pendent targeting ligands A_{b} covalently linked to one or more liposome components.

In another embodiment, conjugates A_{b}-X where X is an antigen or a compound capable of altering progenitor cell function, can be administered in combination with a cytotoxic compound. The cytotoxic compounds listed above are among the compounds suitable for this purpose.

In another method of treatment embodiment, the group X in the targeted conjugate A_{b}-X, comprises an antigen (i.e., a compound that elicits an immune response in vivo), the ligand-antigen conjugates being effective to “label” the population of progenitor cells responsible for disease pathogenesis in the patient suffering from the disease for specific elimination by an endogenous immune response or by co-administered antibodies. The use of ligand-antigen conjugates in the method of treatment described herein works to enhance an immune response-mediated elimination of the progenitor cell population that expresses the ligand receptor. Such elimination can be effected through an endogenous immune response or by a passive immune response effected by co-administered antibodies.

The methods of treatment involving the use of ligand-antigen conjugates are described in U.S. Patent Application Publications Nos. US 2001/0031252 A1 and US 2002/0192157 A1 and PCT Publication No. PCT/US2004/014097, each incorporated herein by reference.

The endogenous immune response can include a humoral response, a cell-mediated immune response, and any other immune response endogenous to the host animal, including complement-mediated cell lysis, antibody-dependent cell-mediated cytotoxicity (ADCC), antibody opsonization leading to phagocytosis, clustering of receptors upon antibody binding resulting in signaling of apoptosis, antiproliferation, or differentiation, and direct immune cell recognition of the delivered antigen (e.g., a hapten). It is also contemplated that the endogenous immune response may employ the secretion of cytokines that regulate such processes as the multiplication, differentiation, and migration of immune cells. The endogenous immune response may include the participation of such immune cell types as B cells, T cells, including helper and cytotoxic T cells, macrophages, natural killer cells, neutrophils, LAK cells, and the like.

The humoral response can be a response induced by such processes as normally scheduled vaccination, or active immunization with a natural antigen or an unnatural antigen or hapten, e.g., fluorescein isothiocyanate (FITC), with the unnatural antigen inducing a novel immunity. Active immunization involves multiple injections of the unnatural antigen or hapten scheduled outside of a normal vaccination regimen to induce the novel immunity. The humoral response may also result from an innate immunity where the host animal has a natural preexisting immunity, such as an immunity to α-galactosyl groups.

Alternatively, a passive immunity may be established by administering antibodies to the host animal such as natural antibodies collected from serum or monoclonal antibodies that may or may not be genetically engineered antibodies, including humanized antibodies. The utilization of a particular amount of an antibody reagent to develop a passive immunity, and the use of a ligand-antigen conjugate wherein the passively administered antibodies are directed to the antigen, would provide the advantage of a standard set of reagents to be used in cases where a patient's preexisting antibody titer to potential antigens is not therapeutically useful. The passively administered antibodies may be “co-administered” with the ligand-antigen conjugate, and co-administration is defined as administration of antibodies at a time prior to, at the same time as, or at a time following administration of the ligand-antigen conjugate.

The preexisting antibodies, induced antibodies, or passively administered antibodies will be redirected to the progenitor cells by preferential binding of the ligand-antigen conjugates to the progenitor cell populations, and such pathogenic cells are killed by complement-mediated lysis, ADCC, antibody-dependent phagocytosis, or antibody clustering of receptors. The cytotoxic process may also involve other types of immune responses, such as cell-mediated immunity.

Acceptable antigens for use in preparing the conjugates used in the method of treatment described herein are antigens that are capable of eliciting antibody production in a host animal or that have previously elicited antibody production in a host animal, resulting in a preexisting immunity, or that constitute part of the innate immune system. Alternatively, antibodies directed against the antigen may be administered to the host animal to establish a passive immunity. Suitable antigens for use in the invention include antigens or antigenic peptides against which a preexisting immunity has developed via normally scheduled vaccinations or prior natural exposure to such agents such as polio virus, tetanus, typhus, rubella, measles, mumps, pertussis, tuberculosis and influenza antigens, and α-galactosyl groups. In such cases, the ligand-antigen conjugates will be used to redirect a previously acquired humoral or cellular immunity to a population of progenitor cells in the host animal for elimination of the progenitor cells.

Other suitable immunogens include antigens or antigenic peptides to which the host animal has developed a novel immunity through immunization against an unnatural antigen or hapten, for example, fluorescein isothiocyanate (FITC) or dinitrophenyl, and antigens against which an innate immunity exists, for example, super antigens and muramyl dipeptide.

The progenitor cell-binding ligands and antigens, cytotoxic agents, compounds capable of altering progenitor cell function, or imaging agents, as the case may be in forming conjugates for use in accordance with the methods described herein can be conjugated by using any art-recognized method for forming a complex. This can include covalent, ionic, or hydrogen bonding of the ligand to the antigen, either directly or indirectly via a linking group such as a divalent linker. The conjugate is typically formed by covalent bonding of the ligand to the targeted entity through the formation of amide, ester or imino bonds between acid, aldehyde, hydroxy, amino, or hydrazo groups on the respective components of the complex or, for example, by the formation of disulfide bonds. Methods of linking binding ligands to antigens, cytotoxic agents, compounds capable of altering progenitor cell function, or quantifiable markers are described in U.S. Patent Application Publication No. US 2005/0002942-A1 and PCT Publication No. WO 2006/012527, each incorporated herein by reference.

Alternatively, as mentioned above, the ligand complex can be one comprising a liposome wherein the targeted entity (that is, the quantifiable marker, or the antigen, cytotoxic agent or progenitor cell function-altering agent) is contained within a liposome which is itself covalently linked to the binding ligand. Other nanoparticles, dendrimers, derivatizable polymers or copolymers that can be linked to therapeutic or quantifiable markers useful in the treatment and diagnosis of progenitor cell-worsened diseases can also be used in targeted conjugates.

In one embodiment of the invention the ligand is folic acid, an analog of folic acid, or any other folate receptor binding molecule, and the folate ligand is conjugated to the targeted entity by a procedure that utilizes trifluoroacetic anhydride to prepare γ-esters of folic acid via a pteroyl azide intermediate. This procedure results in the synthesis of a folate ligand, conjugated to the targeted entity only through the γ-carboxy group of the glutamic acid groups of folate. Alternatively, folic acid analogs can be coupled through the α-carboxy moiety of the glutamic acid group or both the α and γ carboxylic acid entities.

The therapeutic methods described herein can be used to slow the progress of disease completely or partially. Alternatively, the therapeutic methods described herein can eliminate or prevent reoccurrence of the disease state.

The conjugates used in accordance with the methods described herein of the formula A_{b}-X are used in one aspect to formulate therapeutic or diagnostic compositions, for administration to a patient, wherein the compositions comprise effective amounts of the conjugate and an acceptable carrier therefor. Typically such compositions are formulated for parenteral use. The amount of the conjugate effective for use in accordance with the methods described herein depends on many parameters, including the nature of the disease being treated or diagnosed, the molecular weight of the conjugate, its route of administration and its tissue distribution, and the possibility of co-usage of other therapeutic or diagnostic agents. The effective amount to be administered to a patient is typically based on body surface area, patient weight and physician assessment of patient condition. An effective amount can range from about to 1 ng/kg to about 1 mg/kg, more typically from about 1 μg/kg to about 500 μg/kg, and most typically from about 1 μg/kg to about 100 μg/kg.

Any effective regimen for administering the ligand conjugates can be used. For example, the ligand conjugates can be administered as single doses, or they can be divided and administered as a multiple-dose daily regimen. Further, a staggered regimen, for example, one to three days per week can be used as an alternative to daily treatment, and such an intermittent or staggered daily regimen is considered to be equivalent to every day treatment and within the scope of this disclosure. In one embodiment, the patient is treated with multiple injections of the ligand conjugate wherein the targeted entity is an antigen or a cytotoxic agent or a compound capable of altering progenitor cell function to eliminate the population of pathogenic progenitor cells. In one embodiment, the patient is treated, for example, injected multiple times with the ligand conjugate at, for example, 12-72 hour intervals or at 48-72 hour intervals. Additional injections of the ligand conjugate can be administered to the patient at intervals of days or months after the initial injections, and the additional injections prevent recurrence of disease. Alternatively, the ligand conjugates may be administered prophylactically to prevent the occurrence of disease in patients known to be disposed to development of disease states worsened by progenitor cells. In one embodiment, more than one type of ligand conjugate can be used, for example, the host animal may be pre-immunized with fluorescein isothiocyanate and dinitrophenyl and subsequently treated with fluorescein isothiocyanate and dinitrophenyl linked to the same or different targeting ligands in a co-dosing protocol.

The ligand conjugates are administered in one aspect parenterally and most typically by intraperitoneal injections, subcutaneous injections, intramuscular injections, intravenous injections, intradermal injections, or intrathecal injections. The ligand conjugates can also be delivered to a patient using an osmotic pump. Examples of parenteral dosage forms include aqueous solutions of the conjugate, for example, a solution in isotonic saline, 5% glucose or other well-known pharmaceutically acceptable liquid carriers such as alcohols, glycols, esters and amides. The parenteral compositions for use in accordance with this invention can be in the form of a reconstitutable lyophilizate comprising the one or more doses of the ligand conjugate. In another aspect, the ligand conjugates can be formulated as one of any of a number of prolonged release dosage forms known in the art such as, for example, the biodegradable carbohydrate matrices described in U.S. Pat. Nos. 4,713,249; 5,266,333; and 5,417,982, the disclosures of which are incorporated herein by reference. The ligand conjugates can also be administered topically such as in an ointment or a lotion, for example, for treatment of inflammations of the skin.

In any of the embodiments discussed above, the progenitor cells can be activated cells or other cell populations that augment or cause disease states. The following examples are illustrative embodiments only and are not intended to be limiting.

### Example 1

### Materials

Fmoc-protected amino acid derivatives, trityl-protected cysteine 2-chlorotrityl resin (H-Cys(Trt)-2-ClTrt resin #04-12-2811), Fmoc-lysine(4-methyltrityl) wang resin, 2-(1H-benzotriaxol-1-yl)-1,1,3,3-tetramethyluronium hexafluorophosphage (HBTU) and N-hydroxybenzotriazole were purchased from Novabiochem (La Jolla, Calif.). N¹⁰-trifluoroacetylpteroic acid was purchased from Sigma, St. Louis, Mo. Piperidine, DIPEA (diisopropylethylamine), Rhodamine B isothiocyanate (Rd-ITC) and triisopropyl saline (TIPS) were from Aldrich (Milwaukee). Anti-mouse antibodies were purchased from Caltag Laboratories, Burlingame, Calif. The following anti-mouse antibodies were purchased from Caltag Laboratories: CD11b, CD16.2/32.2, CD23, CD44, CD49d, CD62L, CD69, CD80, CD86, F4/80, Ly-6C/G, I-A^{b} MHC Class II and Streptavidin secondary fluorescence tag. The anti-mouse CD8a, CD133, and Flk-1 antibodies were purchased from eBioscience (San Diego, Calif.). The anti-mouse CD115 antibody was purchased from Serotec (Raleigh, N.C.). The anti-mouse CD14 and CD25 antibodies were purchased from Becton Dickinson (Franklin Lakes, N.J.). Folate-R-Phycoerytherin, Folate-AlexaFluor 488, Folate-Texas Red, and Folate-Fluorescein and Folate-cysteine were synthesized as described. Folate-FITC was provided by Endocyte, Inc.

### Example 2

### Synthesis of Folate-Cysteine

Standard Fmoc peptide chemistry was used to synthesize folate-cysteine with the cysteine attached to the γ-COOH of folic acid. The sequence Cys-Glu-Pteroic acid (Folate-Cys) was constructed by Fmoc chemistry with HBTU and N-hydroxybenzotriazole as the activating agents along with diisopropyethylamine as the base and 20% piperidine in dimethylformamide (DMF) for deprotection of the Fmoc groups. An α-t-Boc-protected N-α-Fmoc-L-glutamic acid was linked to a trityl-protected Cys linked to a 2-Chlorotrityl resin. N¹⁰-trifluoroacetylpteroic acid was then attached to the γ-COOH of Glu. The Folate-Cys was cleaved from the resin using a 92.5% trifluoroacetic acid-2.5% water-2.5% triisopropylsilane-2.5% ethanedithio solution. Diethyl ether was used to precipitate the product, and the precipitant was collected by centrifugation. The product was washed twice with diethyl ether and dried under vacuum overnight. To remove the N¹⁰-trifluoracetyl protecting group, the product was dissolved in a 10% ammonium hydroxide solution and stirred for 30 min at room temperature. The solution was kept under a stream of nitrogen the entire time in order to prevent the cysteine from forming disulfides. After 30 minutes, hydrochloric acid was added to the solution until the compound precipitated. The product was collected by centrifugation and lyophilized. The product was analyzed and confirmed by mass spectroscopic analysis (MW 544, M⁺545).

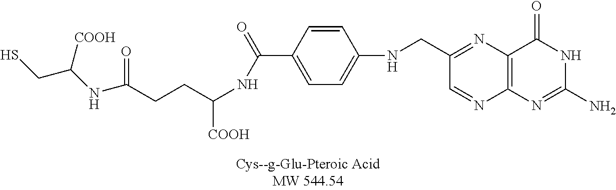 [SMILES: Nc1nc2ncc(CNc3ccc(C(=O)NC(CCC(=O)NC(CS)C(=O)O)C(=O)O)cc3)nc2c(=O)[nH]1]

### Example 3

### Synthesis of Folate-Cys-AlexaFluor 488

AlexaFluor 488 C₅-maleimide (Molecular Probes, Eugene, Oreg.) was dissolved in dimethyl sulfoxide (DMSO) (0.5 mg in 50 μl DMSO). A 1.5 molar equivalent (0.57 mg) of Folate-Cys was added to the solution and mixed for 4 hours at room temperature. Folate-Cys-AlexaFluor 488 (Folate-AlexaFluor) was purified by reverse-phase HPLC on a C18 column at a flow rate of 1 ml/min. The mobile phase, consisting of 10 mM NH₄HCO₃ buffer, pH 7.0 (eluent A) and acetonitrile (eluent B), was maintained at a 99:1 A:B ratio for the first minute and then changed to 1:99 A:B in a linear gradient over the next 29 minutes. Folate-Cys-AlexaFluor 488 eluted at 20 minutes. The product was confirmed by mass spectroscopy and the biologic activity was confirmed by fluorescence measurement of its binding to cell surface folate receptors on folate receptor positive M109 cells in culture.

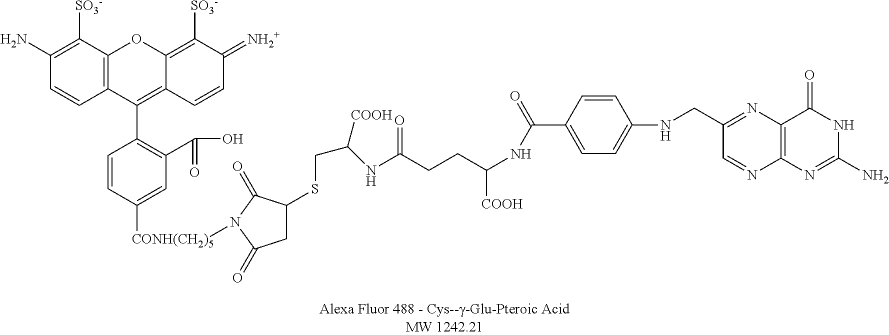

### Example 4

### Synthesis of Folate-Cys-Texas Red

Texas Red C₂-maleimide (Molecular Probes, Eugene, Oreg.) was dissolved in dimethyl sulfoxide (DMSO) (1 mg in 200 μl DMSO). A 1.4 molar equivalent (1 mg) of Folate-Cys was added to the solution and mixed for 4 hours at room temperature. Folate-Cys-Texas Red (Folate-Texas Red) was purified by reverse-phase HPLC on a C18 column at a flow rate of 1 ml/min. The mobile phase, consisting of 10 mM NH₄HCO₃ buffer, pH 7.0 (eluent A) and acetonitrile (eluent B), was maintained at a 99:1 A:B ratio for the first five minutes and then changed to 70:30 A:B in a linear gradient over the next 30 minutes followed by a 1:99 A:B linear gradient over the last 15 minutes. Folate-Cys-Texas Red eluted as two isomer peaks at 44.5 and 45.8 minutes. The product was confirmed by mass spectroscopy and the biologic activity was confirmed by fluorescence measurement of its binding to cell surface folate receptors on folate receptor positive M109 cells in culture.

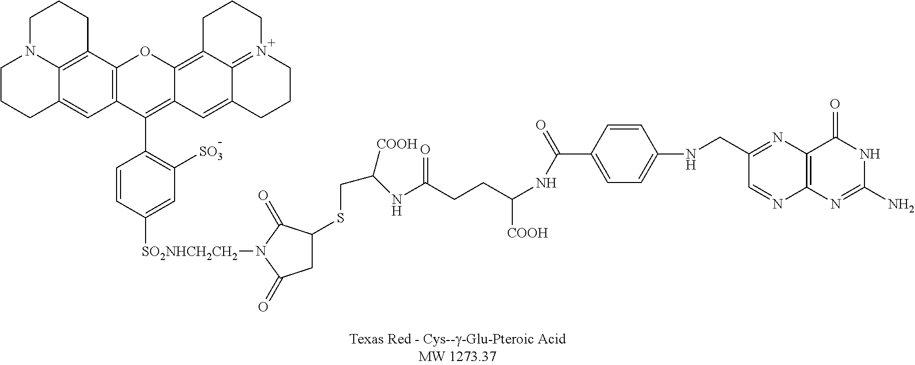

### Example 5

### Synthesis of Folate-Lys-Oregon Green 514

Standard Fmoc peptide chemistry was used to synthesize a folate peptide linked to Oregon Green (Molecular Probes, Eugene, Oreg.) attached to the γ-COOH of folic acid. The sequence Lys-Glu-Pteroic acid (Folate-Cys) was constructed by Fmoc chemistry with HBTU and N-hydroxybenzotriazole as the activating agents along with diisopropyethylamine as the base and 20% piperidine in dimethylformamide (DMF) for deprotection of the Fmoc groups. An α-t-Boc-protected N-α-Fmoc-L-glutamic acid followed by a N¹⁰-trifluoroacetylpteroic acid was linked to a Fmoc-protected lysine wang resin containing a 4-methyltrityl protecting group on the ε-amine. The methoxytrityl protecting group on the ε-amine of lysine was removed with 1% trifluoroacetic acid in dichloromethane to allow attachment of Oregon Green (Folate-Oregon Green). A 1.5 molar equivalent of Oregon Green carboxylic acid, succinimidyl ester was reacted overnight with the peptide and then washed thoroughly from the peptide resin beads. The Folate-Oregon Green was then cleaved from the resin with a 95% trifluoroacetic acid-2.5% water-2.5% triisopropylsilane solution. Diethyl ether was used to precipitate the product, and the precipitant was collected by centrifugation. The product was washed twice with diethyl ether and dried under vacuum overnight. To remove the N¹⁰-trifluoracetyl protecting group, the product was dissolved in a 10% ammonium hydroxide solution and stirred for 30 min at room temperature. The product was precipitated with combined isopropanol and ether, and the precipitant was collected by centrifugation.

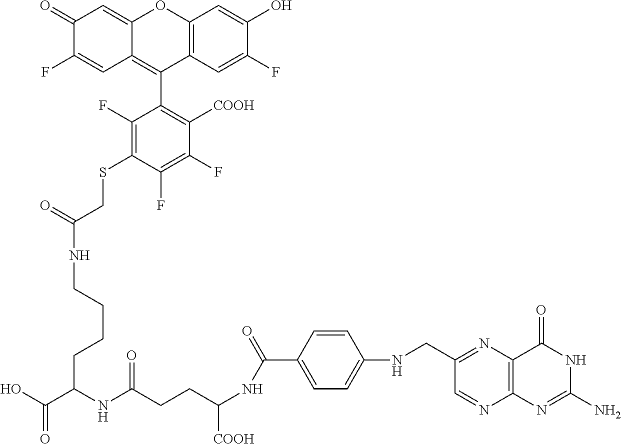 [SMILES: Nc1nc2ncc(CNc3ccc(C(=O)NC(CCC(=O)NC(CCCCNC(=O)CSc4c(F)c(F)c(C(=O)O)c(-c5c6cc(F)c(=O)cc-6oc6cc(O)c(F)cc56)c4F)C(=O)O)C(=O)O)cc3)nc2c(=O)[nH]1]

### Example 6

### Synthesis of Folate-R-Phycoerythrin

Folate-phycoerythrin was synthesized by following a procedure published by Kennedy M. D. et al. in Pharmaceutical Research, Vol. 20(5); 2003. Briefly, a 10-fold excess of folate-cysteine was added to a solution of R-phycoerythrin pyridyldisulfide (Sigma, St. Louis, Mo.) in phosphate buffered saline (PBS), pH 7.4. The solution was allowed to react overnight at 4° C. and the labeled protein (Mr ˜260 kDa) was purified by gel filtration chromatography using a G-15 desalting column. The folate labeling was confirmed by fluorescence microscopy of M109 cells incubated with folate-phycoerythrin in the presence and absence of 100-fold excess of folic acid. After a 1-h incubation and 3 cells washes with PBS, the treated cells were intensely fluorescent, while the sample in the presence of excess folic acid showed little cellular fluorescence.

### Example 7

### Synthesis of Folate-Fluorescein

Folate-FITC was synthesized as described by Kennedy, M. D. et al. in Pharmaceutical Research, Vol. 20(5); 2003.

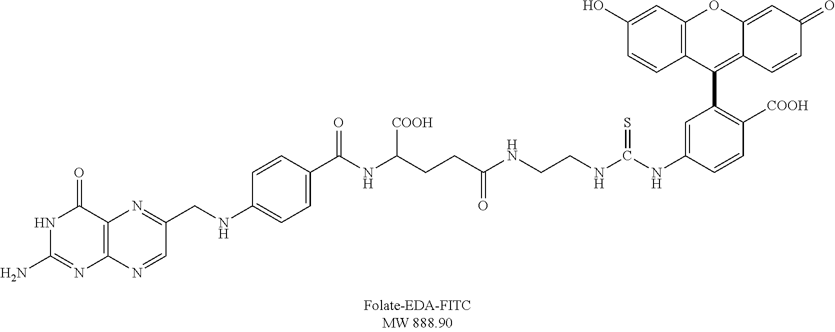

### Example 8

### Synthesis of Folate-D-R-D-D-C-Prednisolone

Standard Fmoc peptide chemistry was used to synthesize folate-aspartate-arginine-aspartate-aspartate-cysteine (Folate-Asp-Arg-Asp-Asp-Cys, Folate-D-R-D-D-C) with the amino acid spacer attached to the γ-COOH of folic acid. The sequence Cys-Asp-Asp-Arg-Asp-Glu-Pteroic acid (Folate-Asp-Arg-Asp-Asp-Cys) was constructed by Fmoc chemistry with HBTU and N-hydroxybenzotriazole as the activating agents along with diisopropyethylamine as the base and 20% piperidine in dimethylformamide (DMF) for deprotection of the Fmoc groups. Fmoc-D-Asp(OtBu)-OH was linked to a trityl-protected Cys linked to a 2-Chlorotrityl resin. A second Fmoc-D-Asp(OtBu)-OH followed by Fmoc-Arg(Pbf)-OH, Fmoc-D-Asp(OtBu)-OH and Fmoc-Glu-OtBu were added successively to the resin. N¹⁰-trifluoroacetylpteroic acid was then attached to the γ-COOH of Glu. The Folate-Asp-Arg-Asp-Asp-Cys was cleaved from the resin using a 92.5% trifluoroacetic acid-2.5% water-2.5% triisopropylsilane-2.5% ethanedithio solution. Diethyl ether was used to precipitate the product, and the precipitant was collected by centrifugation. The product was washed twice with diethyl ether and dried under vacuum overnight. To remove the N¹⁰-trifluoracetyl protecting group, the product was dissolved in a 10% ammonium hydroxide solution and stirred for 30 min at room temperature. The solution was kept under a stream of nitrogen the entire time in order to prevent the cysteine from forming disulfides. After 30 minutes, hydrochloric acid was added to the solution until the compound precipitated. The product was collected by centrifugation and lyophilized. The product was analyzed and confirmed by mass spectroscopic analysis (MW 1046).

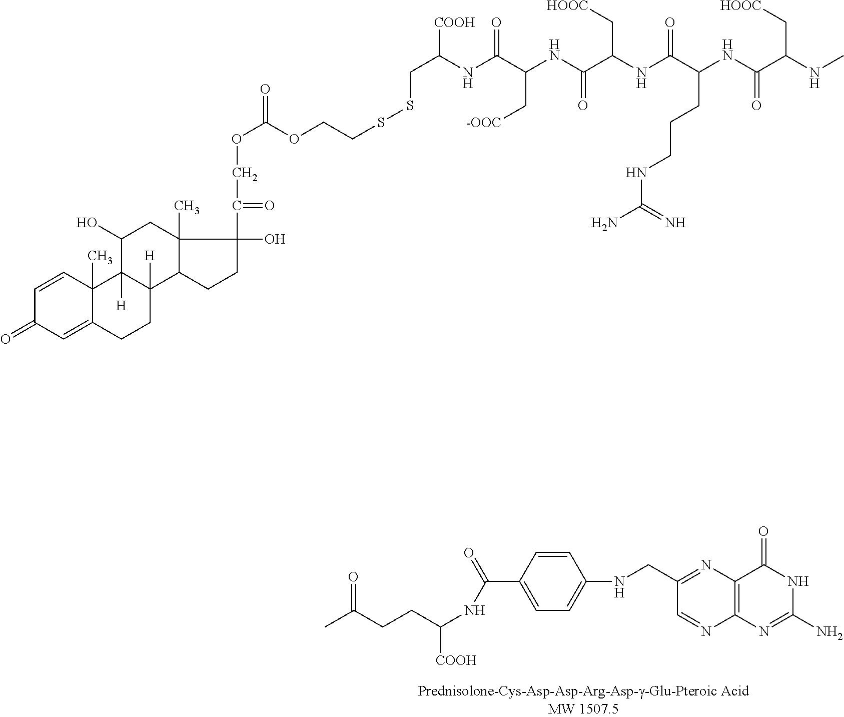

### Example 9

### Synthesis of Folate-Indomethacin

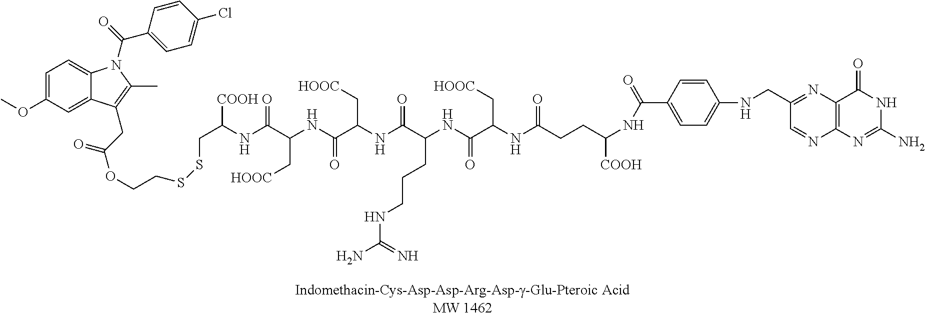 [SMILES: COc1ccc2c(c1)c(CC(=O)OCCSSCC(NC(=O)C(CC(=O)O)NC(=O)C(CC(=O)O)NC(=O)C(CCCNC(=N)N)NC(=O)C(CC(=O)O)NC(=O)CCC(NC(=O)c1ccc(NCc3cnc4nc(N)[nH]c(=O)c4n3)cc1)C(=O)O)C(=O)O)c(C)n2C(=O)c1ccc(Cl)cc1]

2-(2-Pyridyldithio)ethanol was synthesized by dissolving 1.5 equivalents of Aldrithiol (Sigma, St. Louis, Mo.) with 6 equivalents of 4-dimethylaminopyridine (DMAP) in dichloromethane (DCM). The solution was purged with nitrogen and 1 equivalent of mercaptoethanol was added dropwise to the Aldrithiol solution over the course of 15 minutes. The reaction proceeded at room temperature for 30 minutes at which time no odor of mercaptoethanol remained. The reaction was diluted 100-fold with DCM and 5 g of activated carbon was added per gram of Aldrithiol. The reaction mixture was filtered and the solvent removed. The mixture was resuspended in 70:30 (Petroleum ether:Ethylacetate (EtOAc)) and purified by flash chromatography on a 60 Å silica gel column. The product was monitored by thin layer chromatography and collected.

Folate-indomethacin was synthesized following a modified method published by Kalgutkar et al. in the Journal of Med. Chem. 2000, 43; 2860-2870 where the anti-inflammatory (indomethacin) was linked through an ester bond with the 2-(2-Pyridyldithio)ethanol. Briefly, 1 equivalent of indomethacin was dissolved in DCM along with 0.08 equivalents DMAP, 1.1 equivalents 2-(2-Pyridyldithio) ethanol and 1.1 equivalents 1,3-dicyclohexyl-carbodiimide. The reaction proceeded at room temperature for 5 hours. The reaction was purified by chromatography on silica gel (EtOAc:hexanes, 20:80). One equivalent of the purified compound was dissolved in DMSO and to it were added 1.5 equivalents of the folate-Asp-Arg-Asp-Asp-Cys peptide. The resulting solution was reacted for 3 hours at room temperature followed by purification using a HPLC reverse-phase C18 column at a flow rate of 1 ml/min. The mobile phase, consisting of 10 mM NH₄HCO₃ buffer, pH 7.0 (eluent A) and acetonitrile (eluent B), was maintained at a 99:1 A:B ratio for the first five minutes and then changed to 70:30 A:B in a linear gradient over the next 30 minutes. The recovered final product was confirmed by mass spectrometry.

### Example 10

### Synthesis of Folate-Diclofenac

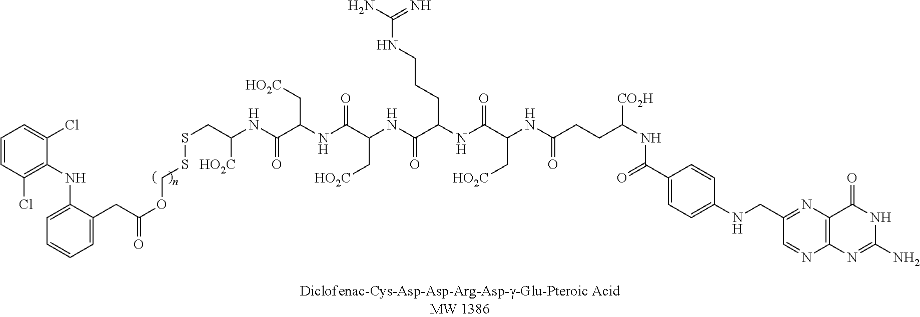

Folate-diclofenac was synthesized by the method described in Example 9 except that diclofenac was used in place of indomethicin. In various embodiments, n=1, 2, or 3, and where n is illustratively 2.

### Example 11

### Synthesis of Folate-Cys-Prednisolone

The folate glucocorticoid conjugate of prednisolone was prepared as follows. A 1.1 molar equivalent of prednisone was dissolved in tetrahydrofuran (THF). In a separate vial, a 0.7 molar equivalent of dimethylaminopyridine, 1 molar equivalent of tri(hydroxyethyl)amine and 1 molar equivalent of the linker (synthesis described in PCT Publication No. WO 2006/012527, incorporated herein by reference) were dissolved in dichloromethane. An approximately equal volume of both solutions were combined, mixed and reacted at room temperature for 4 hours. The reaction was monitored by thin layer chromatography using 40:10:1 (Dichloromethane:Acetonitrile:Methanol). The product had an R_{f}=0.52. The product was purified on a silica column (Silica 32-63, 60 Å) using the same ratio of solvents. The recovered product was dried in preparation for conjugation to a folate-peptide. The derivatized glucocorticoid was dissolved in DMSO, to which was added a 1.5 molar equivalent of either the folate-cys or folate-Asp-Arg-Asp-Asp-Cys peptide. The resulting solution was reacted for 3 hours at room temperature followed by purification using a HPLC reverse-phase C18 column at a flow rate of 1 ml/min. The mobile phase, consisting of 10 mM NH₄HCO₃ buffer, pH 7.0 (eluent A) and acetonitrile (eluent B), was maintained at a 99:1 A:B ratio for the first minute and then changed to 1:99 A:B in a linear gradient over the next 39 minutes. The folate-glucocorticoid conjugate eluted at approximately 26 minutes. The recovered final product was confirmed by mass spectrometry.

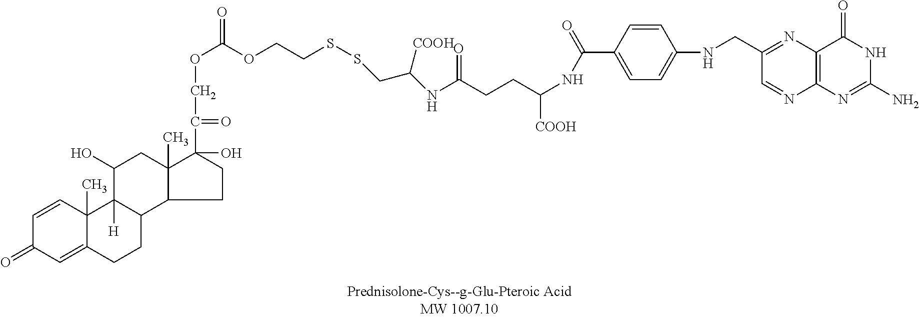

### Example 12

### Synthesis of Folate-Cys-Dexamethasone

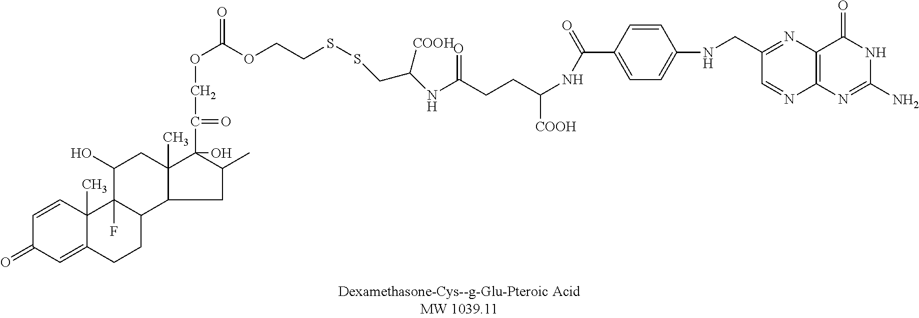

Folate-cys-dexamethasone was synthesized by a procedure similar to that described in Example 11 except that the glucocorticoid was dexamethasone.

### Example 13

### Synthesis of Folate-Cys-Flumethasone

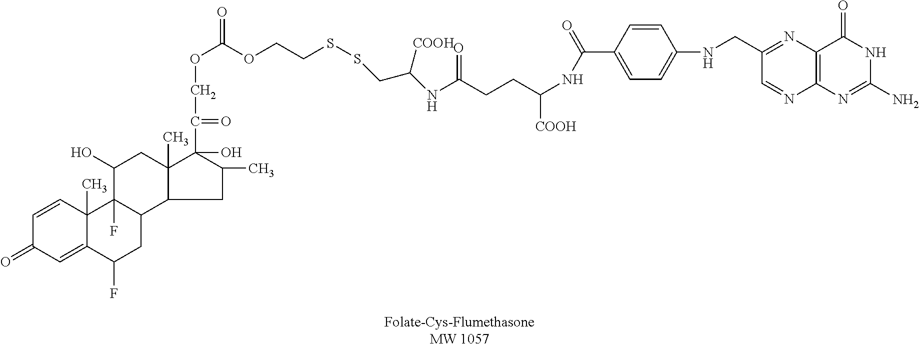

Folate-cys-flumethasone was synthesized by a procedure similar to that described in Example 11 except that the glucocorticoid was flumethasone.

### Example 14

### Isolation of Folate-Receptor-Positive Endothelial Progenitor Cells

Female 6- to 8-week-old BALB/c mice were injected in the peritoneal cavity with either Complete Freund's Adjuvant (CFA; 50-100 μL), Pseudomonas aeruginosa (1×10⁷ CFU (colony forming units)), or Yersinia enterocolitica (1×10⁶ CFU). Cells were isolated from the peritoneal cavity by lavage with 8 mL of sterile phosphate-buffered saline (PBS) 2-4 days later. The cells were pelleted by centrifugation (400×g, 10 minutes at room temperature) and resuspended in folate-deficient RPMI-1640 media (FD-RPMI; Gibco) containing 10% heat-inactivated fetal bovine serum (FBS), penicillin (100 IU/mL) and streptomycin (100 μg/mL). Peritoneal extracted cells were seeded at densities of 1×10⁶ cells/microcentrifuge tube for antibody and folate conjugate studies.

### Example 15

### Ligand Binding

All binding experiments were conducted on ice or in a 4° C. cold room unless indicated otherwise. All antibody labeling was optimized by titration. Optimal labeling was most often achieved with a 1/1000-1/10,000 dilution of the manufacture's stock antibody solution. After cells were labeled with antibodies, the samples were washed twice with PBS to remove non-specific binding. The samples were then incubated with a 100 nM concentration of folate-FITC for 45 minutes. Competition samples were prepared by pre-incubating the appropriate samples with a 100-fold excess concentration of folic acid (10 μM) for five minutes prior to adding the folate dye conjugate. All samples were analyzed by flow cytometry using a Becton Dickinson FACS Calibur (BD, Franklin Lakes, N.J.).

### Example 16

### Synthesis of Folate Resonance Energy Transfer Reporter

Compound 1 was prepared by following standard Fmoc chemistry on an acid-sensitive trityl resin loaded with Fmoc-L-Cys (Trt)-OH, as described previously (adapted to the shown peptide sequence). The crude compound 1 was purified by HPLC using a VYDAC protein and peptide C18 column. The HPLC-purified 1 was then reacted with tetraethylrhodamine methanethiosulfonate (Molecular Probes, Eugene, Oreg.) in DMSO to afford compound 2, in the presence of diisopropylethylamine (DIPEA). The desired product was isolated from the reaction mixture by preparative HPLC as described above. The final conjugation was performed by mixing excess DIPEA with 2 (in DMSO) followed by addition of BODIPY FL NHS ester (Molecular Probes, Eugene, Oreg.). Compound 3 was then isolated from this reaction mixture by preparative HPLC.

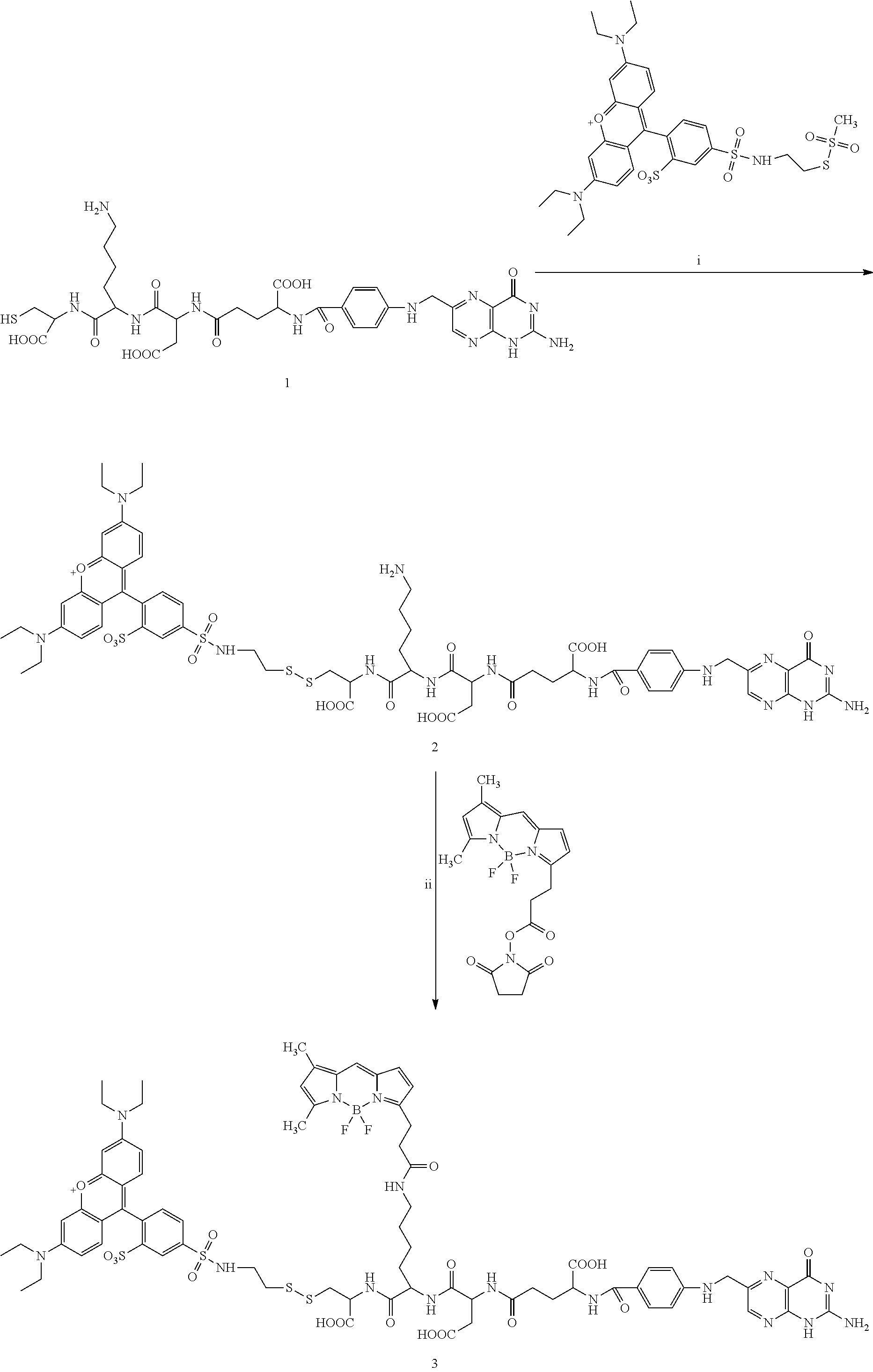

### Example 17

### Laser Imaging

Fluorescence resonance energy transfer (FRET) imaging of progenitor cells to determine uptake of folate-linked markers will be carried out using a confocal microscopy. An Olympus IX-70 inverted microscopy (Olympus, USA) equipped with an Olympus FW300 scanning box and an Olympus 60X/1.2 NA water objective will be used to image the cells. Separate excitation lines and emission filters will be used for each fluorochrome (BODIPY FL, 488 nm (excitation) and 520/40 nm (emission); rhodamine, 543 nm (excitation) and 600/70 nm (emission)). Two laser sources with 543 nm (He—Ne) and 488 nm (Argon) wavelength can be used to excite BODIPY FL and rhodamine separately to obtain two color images when needed. Confocal images can be acquired with a size of 512×512 pixels at 2.7 second scan time and images can be processed using FluoView (Olympus) software.

### Example 18

### Liposome Preparation

Liposomes were prepared following methods by Leamon et al. in Bioconjugate Chemistry 2003, 14, 738-747. Briefly, lipids and cholesterol were purchased from Avanti Polar Lipids (Alabaster, Ala.). Folate-targeted liposomes consisted of 40 mole % cholesterol, either 4 mole % or 6 mole % polyethyleneglycol (Mr˜2000)-derivatized phosphatidylethanolamine (PEG2000-PE, Nektar Ala., Huntsville, Ala.), either 0.03 mole % or 0.1 mole % folate-cysteine-PEG3400-PE and the remaining mole % was composed of egg phosphatidylcholine. Non-targeted liposomes were prepared identically with the absence of folate-cysteine-PEG3400-PE.

Lipids in chloroform were dried to a thin film by rotary evaporation and then rehydrated in PBS containing the drug. Rehydration was accomplished by vigorous vortexing followed by 10 cycles of freezing and thawing. Liposomes were then extruded 10 times through a 50 nm pore size polycarbonate membrane using a high-pressure extruder (Lipex Biomembranes, Vancouver, Canada).

### Example 19

### Synthesis of Folate-Pokeweed

Pokeweed antiviral protein was purchased from Worthington Biochemical Corporation (Lakewood, N.J.). N-succinimidyl-3[2-pyridyldithio]propionate (SPDP; Pierce, Rockford, Ill.) was dissolved in dimethylformamide (9.6 mM). While on ice, a 5 fold molar excess of SPDP (˜170 nmoles) was added to the pokeweed solution (1 mg/ml PBS, MW˜29,000). The resulting solution was gently mixed and allowed to react for 30 minutes at room temperature. The non-conjugated SPDP was removed using a centrifuge molecular weight concentrator (MWCO 10,000) (Millipore, Billerica, Mass.). The resulting protein solution was resuspended in PBS containing 10 mM EDTA to a final volume of 1 mL. Approximately a 60 fold molar excess of folate-Asp-Arg-Asp-Asp-Cys peptide (2000 nmoles) was added to the protein solution and allowed to react for 1 hour. The non-reacted folate-Asp-Arg-Asp-Asp-Cys peptide was removed using the centrifuge concentrators as previously described. The protein was washed twice by resuspending the protein in PBS and repeating the protein concentration by centrifugation.

### Example 20

### Synthesis of Folate-Saporin

The protein saporin was purchased from Sigma (St. Louis, Mo.). Folate-saporin was prepared following folate-protein conjugation methods published by Leamon and Low in The Journal of Biological Chemistry 1992, 267(35); 24966-24971. Briefly, folic acid was dissolved in DMSO and incubated with a 5 fold molar excess of 1-ethyl-3-(3-dimethylaminopropyl)carbodiimide for 30 minutes at room temperature. The saporin was dissolved in 100 mM KH₂PO₄, 100 mM boric acid, pH 8.5. A 10-fold molar excess of the “activated” vitamin was added to the protein solution and the labeling reaction was allowed to proceed for 4 hours. Unreacted material was separated from the labeled protein using a Sephadex G-25 column equilibrated in phosphate-buffered saline, pH 7.4.

### Example 21

### Synthesis of Folate-Momordin and Folate-Gelonin

The proteins momordin and gelonin were purchased from Sigma (St. Louis, Mo.). Folate-cys pyridyldisulfide was prepared by reacting folate-cys with Aldrithiol (Sigma, St. Louis, Mo.). Both proteins were dissolved in 0.1M HEPPS buffer, pH 8.2. A 6-fold molar excess of Trouts reagent (Aldrich St. Louis, Mo.) dissolved in DMSO (16 mM) was added to each protein solution. The solutions were allowed to react for 1 hour at room temperature. Unreacted material was separated from the protein using a Sephadex G-25 column equilibrated in 0.1M phosphate buffer, pH 7.0. Ellmans test for the presence of free thiols were positive for both proteins. While the protein solution was on ice, a 5-fold molar excess of folate-cys pyridyldisulfide dissolved in DMSO was added. The resulting solution was warmed up to room temperature and reacted for 30 minutes. Unreacted material was separated from the labeled protein using a Sephadex G-25 column equilibrated in phosphate-buffered saline, pH 7.4.

### Example 22

### Preparation of Folate-Targeted Clodronate or Prednisolone Phosphate Liposomes

Liposomes were prepared following methods by Leamon et al. in Bioconjugate Chemistry 2003, 14; 738-747. Briefly, lipids and cholesterol were purchased from Avanti Polar Lipids (Alabaster, Ala.). Folate-targeted liposomes consisted of 40 mole % cholesterol, 5 mole % polyethyleneglycol (Mr˜2000)-derivatized phosphatidylethanolamine (PEG2000-PE, Nektar Ala., Huntsville, Ala.), 0.03 mole % folate-cysteine-PEG3400-PE and 54.97 mole % egg phosphatidylcholine. Lipids in chloroform were dried to a thin film by rotary evaporation and then rehydrated in PBS containing either clodronate (250 mg/ml) or prednisolone phosphate (100 mg/ml). Rehydration was accomplished by vigorous vortexing followed by 10 cycles of freezing and thawing. Liposomes were then extruded 10 times through a 50 nm pore size polycarbonate membrane using a high-pressure extruder (Lipex Biomembranes, Vancouver, Canada). The liposomes were separated from unencapsulated clodronate or prednisolone phosphate by passage through a CL4B size exclusion column (Sigma, St. Louis, Mo.) in PBS. Average particle size was between 70 and 100 nm.

### Example 23

### Folate-FITC Binding to Endothelial Progenitor Cells

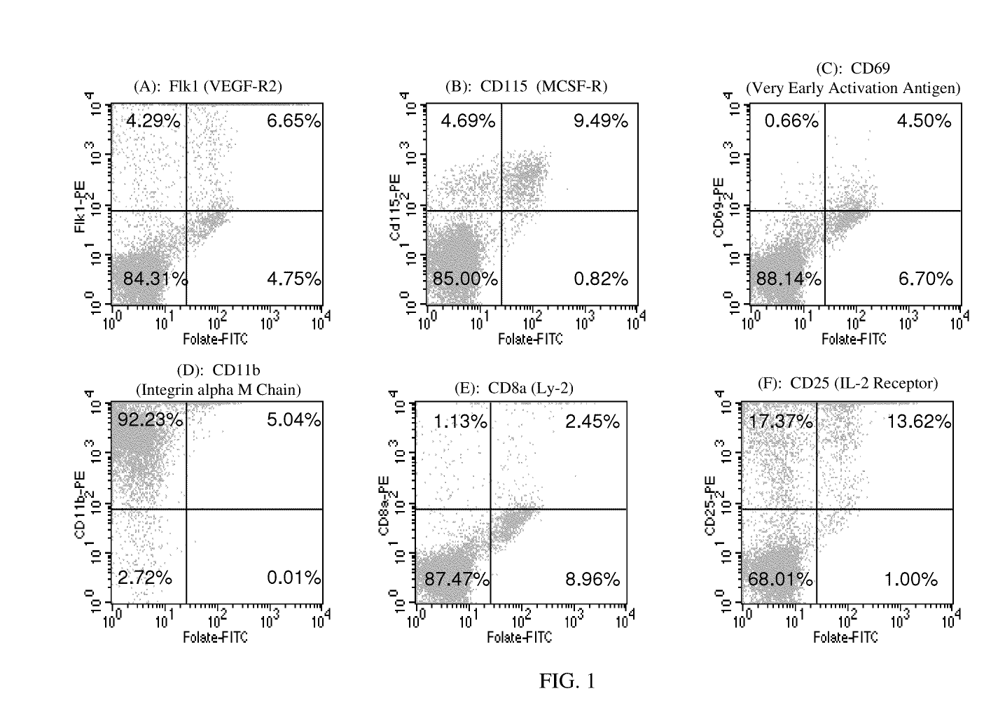

Folate-FITC binding to CD133⁺ Flk1⁺ endothelial progenitor cells and binding of antibodies to Flk1, CD115, CD69, C11b, CD8a, and CD25 markers on endothelial progenitor cells was quantified. Endothelial progenitor cells were isolated as described in Example 14 and folate-FITC and antibody binding and flow cytometry were performed as described in Example 15. As shown in FIG. 1, Flk1, CD115, CD69, CD8a, and CD25 markers are co-expressed with the folate receptor on the progenitor cells.

### Example 24

### Folate-FITC Binding to Endothelial Progenitor Cells

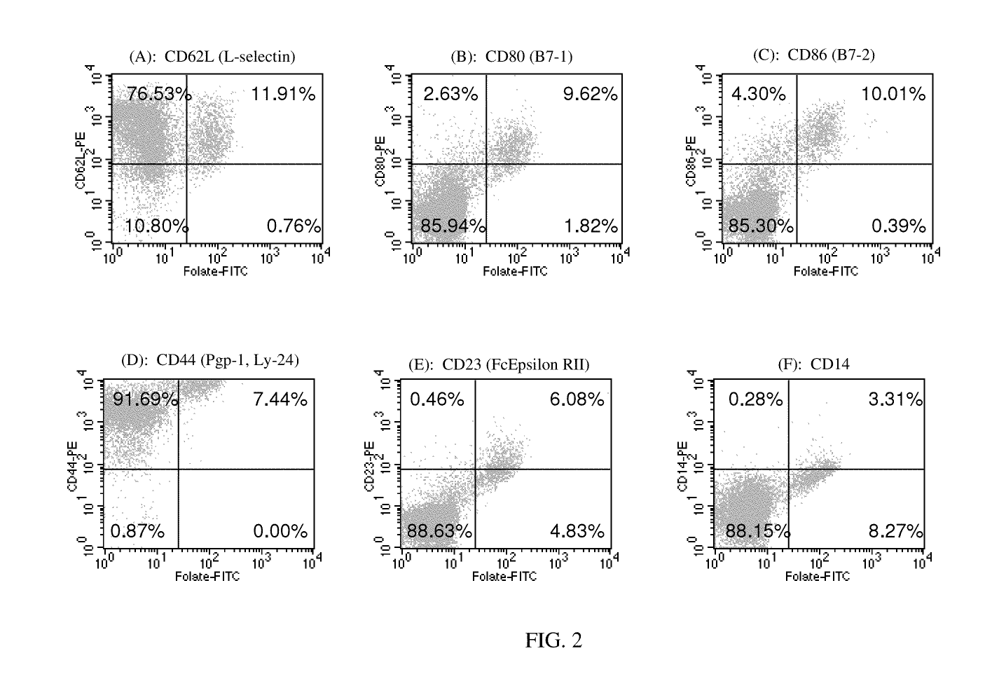

Folate-FITC binding to CD133⁺ Flk1⁺ endothelial progenitor cells and binding of antibodies to CD62L, CD80, CD86, CD44, CD23, and CD14 markers on endothelial progenitor cells was quantified. Endothelial progenitor cells were isolated as described in Example 14 and folate-FITC and antibody binding and flow cytometry were performed as described in Example 15. As shown in FIG. 2, CD62L, CD80, CD86, CD23, and CD14 markers are co-expressed with the folate receptor on CD133⁺ Flk1⁺ endothelial progenitor cells.

### Example 25

### Folate-FITC Binding to Endothelial Progenitor Cells

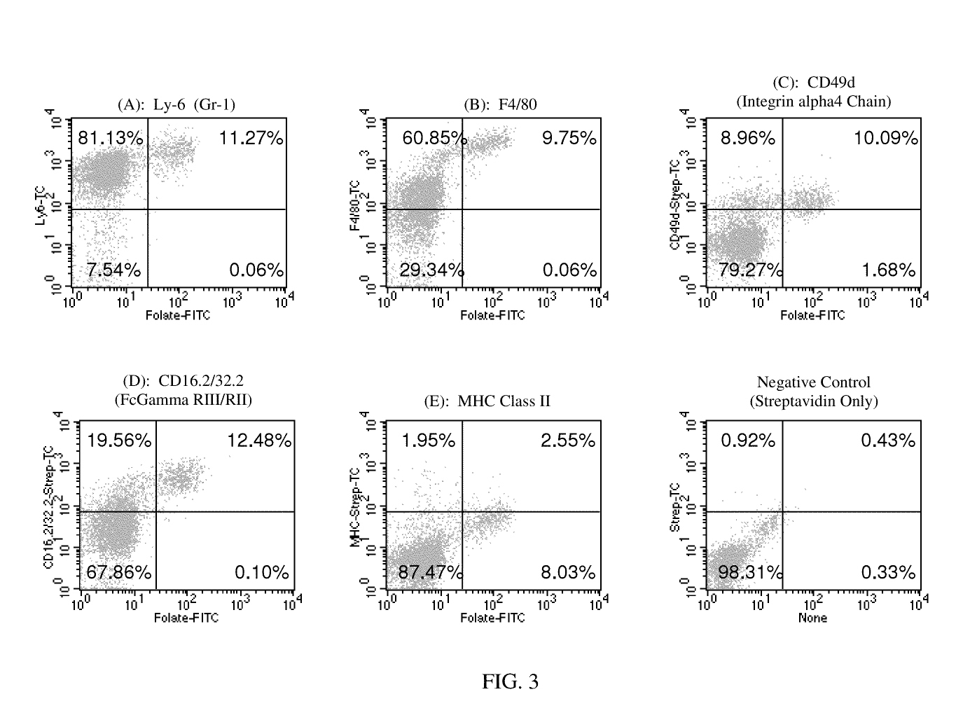

Folate-FITC binding to CD133⁺ Flk1⁺ endothelial progenitor cells and binding of antibodies to Ly-6, F4/80, CD49d, CD16.2/32.2, and MHC Class II markers on endothelial progenitor cells was quantified. Endothelial progenitor cells were isolated as described in Example 14 and folate-FITC and antibody binding and flow cytometry were performed as described in Example 15. As shown in FIG. 3, Ly-6, F4/80, CD49d, and CD16.2/32.2 markers are co-expressed with the folate receptor on the progenitor cells.

### Example 26

### Folate-FITC Binding to Endothelial Progenitor Cells

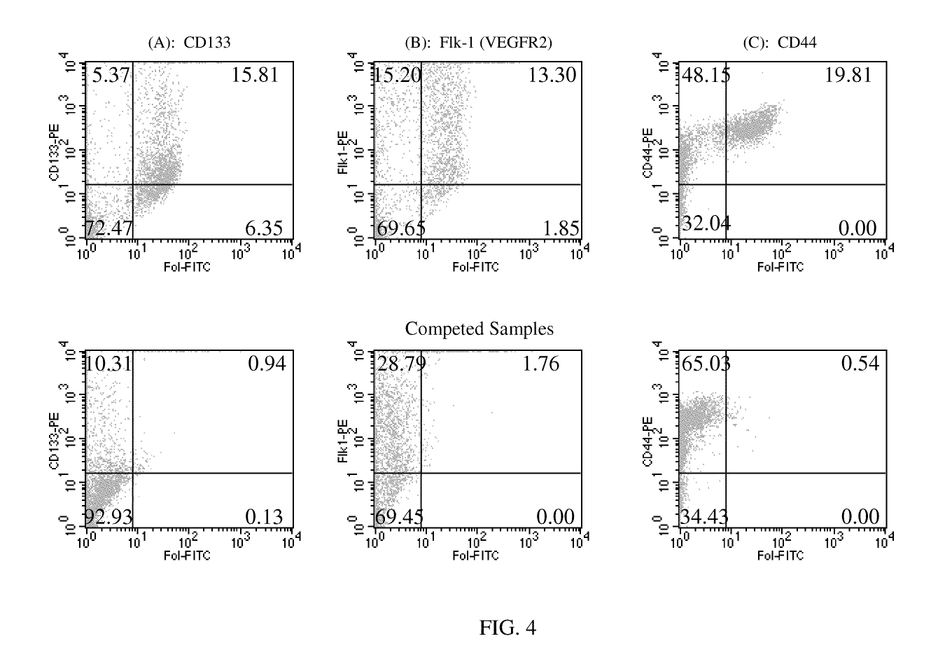

Folate-FITC binding to CD133⁺ Flk1⁺ endothelial progenitor cells and binding of antibodies to CD133, Flk-1, and CD44 markers on endothelial progenitor cells was quantified. Endothelial progenitor cells were isolated as described in Example 14 and folate-FITC and antibody binding and flow cytometry were performed as described in Example 15. As shown in FIG. 4, CD133, Flk-1, and CD44 markers are co-expressed with the folate receptor CD133⁺ Flk1⁺ endothelial progenitor cells. As also shown in FIG. 4, folate-FITC bound to CD133⁺ Flk1⁺ endothelial progenitor cells in the absence of unlabeled folic acid and binding was competed in the presence of a 100-fold excess of unlabeled folic acid.

### Example 27

### Folate-FITC Binding to Endothelial Progenitor Cells

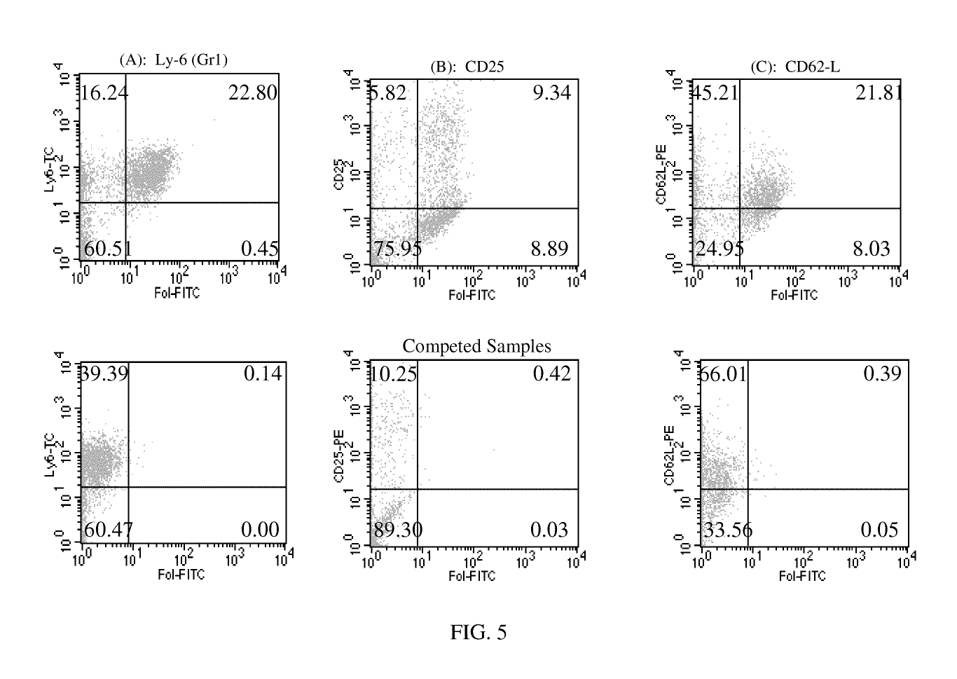

Folate-FITC binding to CD133⁺ Flk1⁺ endothelial progenitor cells and binding of antibodies to Ly-6, CD25, and CD62-L markers on endothelial progenitor cells was quantified. Endothelial progenitor cells were isolated as described in Example 14 and folate-FITC and antibody binding and flow cytometry were performed as described in Example 15. As shown in FIG. 5, Ly-6, CD25, and CD62-L markers are co-expressed with the folate receptor on CD133⁺ Flk1⁺ endothelial progenitor cells. As also shown in FIG. 5, folate-FITC bound to CD133⁺ Flk1⁺ endothelial progenitor cells in the absence of unlabeled folic acid and binding was competed in the presence of a 100-fold excess of unlabeled folic acid.

### Example 28

### Solid Phase Synthesis of Folate Conjugates

The precursor of folate, N¹⁰-TFA-Pteroic acid was synthesized according to standard procedures. Fmoc-Lys(Mtt)-Wang resin was soaked in DMF for 20 minutes with nitrogen bubbling before the reaction. 20% piperidine was added to cleave the Fmoc protective group. 2.5 e.q. Fmoc-Glu-OtBu, HOBT and HBTU, dissolved in DMF, as well as 4e.q. DIPEA were added to the reaction funnel. After 2 hours of nitrogen bubbling at room temperature, the Fmoc cleavage step was repeated with 20% piperidine. 1.5 e.q. N¹⁰-TFA-Pteroic acid and 2.5 e.q. HOBT and HBTU, dissolved in 1:1 DMF/DMSO (dimethylformamide/dimethylsulfoxide), as well as 4 e.q. DIPEA were then added to the reaction for 4 hours with bubbling with nitrogen. The product was then washed with DMF, DCM (dichloromethane), methanol and isopropyl alcohol thoroughly and dried under nitrogen. 1% TFA/DCM (trifluoroacetic acid/dichloromethane) was used to cleave the Mtt (Mtt=4-methyl-trityl) group. 2.5 e.q. Rd-ITC, dissolved in DMF, and 4 e.q. DIPEA were added to the resin and reaction was carried out at room temperature overnight under reduced light conditions. Cleavage of the conjugates was achieved by TFA:TIPS:H₂O (95:2.5:2.5). The crude product was collected by precipitation with cool ether. The crude product was lyophilized overnight. On the second day, the crude product was hydrolyzed using 10% ammonium hydroxide (pH=10) for 45 minutes with nitrogen bubbling. The product was collected by lyophilization. Purification was carried out using preparative HPLC (Rigel).

### Example 29

### Synthesis of Folate Oregon Green 488

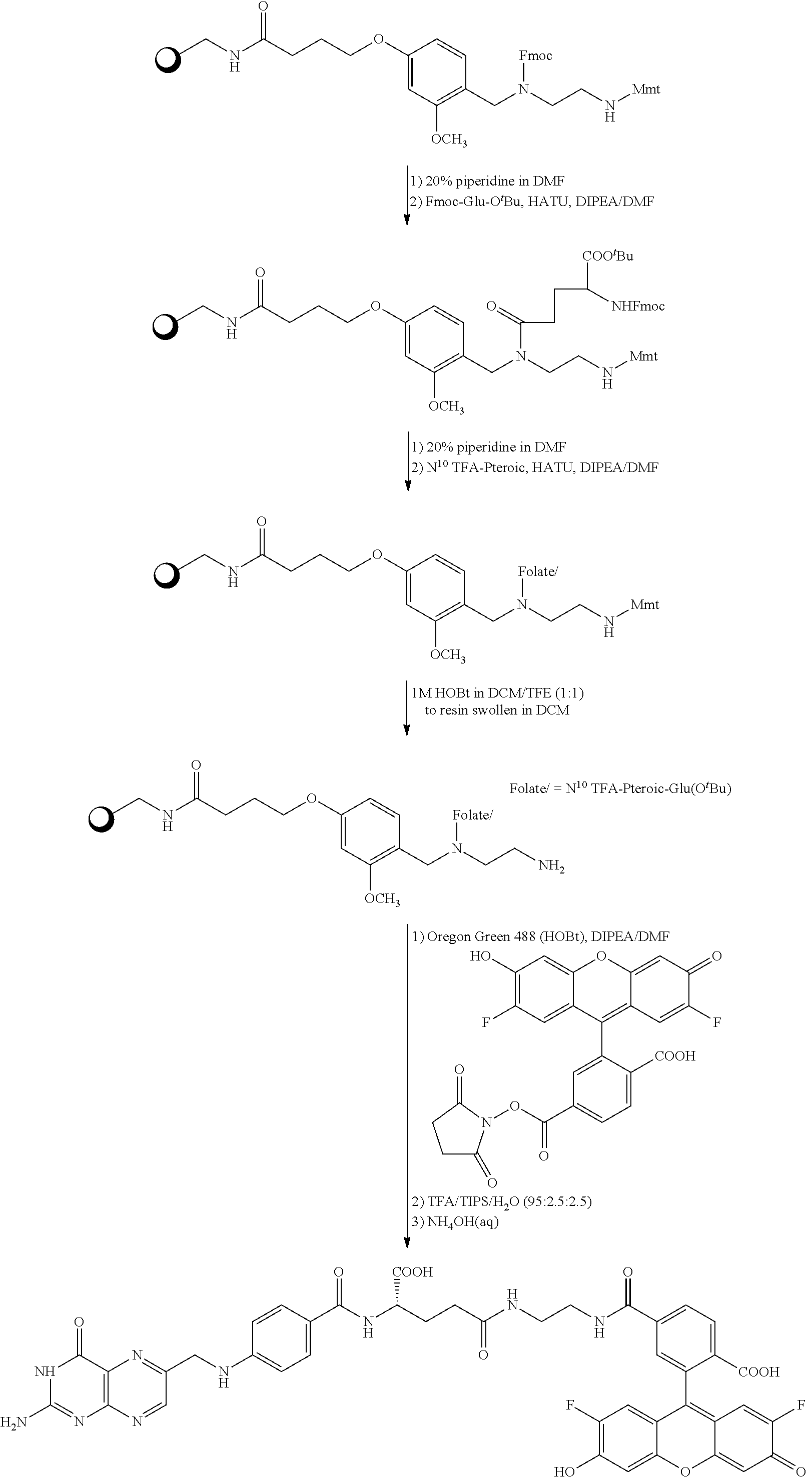

N¹⁰ TFA-Pteroic acid was synthesized as follows. A universal folate resin was synthesized using Universal NovaTag™ resin (Novabiochem; Catalog #04-12-3910). After swelling the resin in DCM (Dichloromethane) for one hour and then with DMF (N,N-Dimethylformamide) for thirty minutes, deprotection of the Fmoc (Fluorenlmethyloxycarbonyl) protecting group was achieved by using a solution of 20% piperidine in DMF. Then Fmoc-Glu-OtBu (three-fold molar excess) was coupled to the deprotected secondary amine using HATU [2-(1H-7-Azabenzotriazol-1-yl)-1,1,3,3-tetramethyl uronium hexafluorophosphate] (three-fold molar excess) and DIPEA (N,N-Diisopropylethylamine) (ten-fold molar excess) in DMF. After thorough washing of this resin, the Fmoc on Glu was removed as described above and N¹⁰-TFA Pteroic acid was coupled using standard Fmoc solid phase peptide synthesis (SPPS) procedures. Next, the pendant Mmt (4-Methoxytrityl) was removed with 1M HOBT (1-Hyroxybenzotriazole) in DCM/TFE (Trifluoroethanol). At this point the resin can be washed with DMF and used immediately for further synthesis or washed sequentially with DCM,DMF and MeOH (Methanol), and dried for later use. To the deprotected, amine reactive universal folate resin, a 1.5-fold molar excess of Oregon Green 488 carboxylic acid succinimidyl ester 6 isomer (P-6149) and a 3-fold molar excess of DIPEA was allowed to react for 12 h at room temperature. The resin was next exhaustively rinsed with DMF, DCM, and methanol and dried for 2 hours. The Folate-Oregon Green 488 was then cleaved from the resin with a 95% trifluoroacetic acid-2.5% water-2.5% triisopropylsilane solution. Diethyl ether was used to precipitate the product, and the precipitant was collected by centrifugation. The product was washed twice with diethyl ether and dried under vacuum overnight. To remove the N¹⁰-trifluoracetyl protecting group, the product was dissolved in a 10% ammonium hydroxide solution and stirred for 30 mM at room temperature. The product was precipitated with combined isopropanol and ether, and the precipitant was collected by centrifugation. The product was purified by reverse-phase HPLC on a C18 column at a flow rate of 1 ml/min. The mobile phase, consisting of 10 mM NH₄HCO₃ buffer, pH 7.0 (eluent A) and acetonitrile (eluent B), was maintained at a 99:1 A:B ratio for the first minute and then changed to 1:99 A:B in a linear gradient over the next 29 minutes. The product was confirmed by MS and NMR.

### Example 30

### Synthesis of Folate Dylight 680

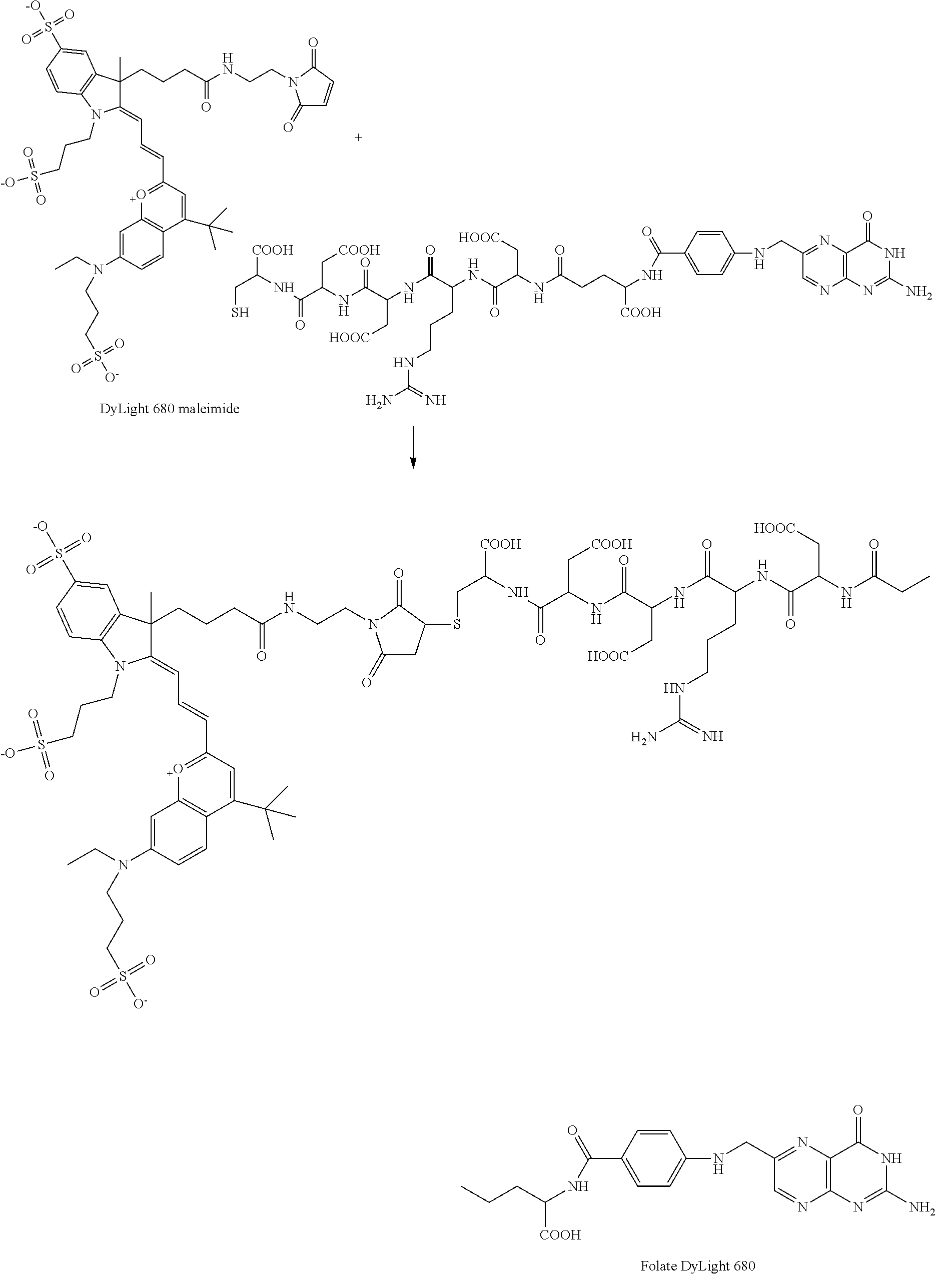

Dylight 680 Maleimide (Pierce) was dissolved in dimethyl sulfoxide (DMSO) (1 mg in 100 uL DMSO). A 3-fold molar excess of Folate-Asp-Arg-Asp-Asp-Cys (synthesized as previously described: Bioorganic & Medicinal Chemistry Letters, Volume 16, Issue 20, 15 Oct. 2006, Pages 5350-5355) was added to the solution and mixed for 4 hours at room temperature. Folate-Dylight 680 was purified by reverse-phase HPLC on a C18 column at a flow rate of 1 ml/min. The mobile phase, consisting of 10 mM NH₄HCO₃ buffer, pH 7.0 (eluent A) and acetonitrile (eluent B), was maintained at a 99:1 A:B ratio for the first minute and then changed to 1:99 A:B in a linear gradient over the next 29 minutes. The product was confirmed by MS and NMR ((C₈₃H₁₀₃N₁₉O₃₀S₄)²⁻; exact Mass: 1973.60; molecular weight: 1975.08; C, 50.47; H, 5.26; N, 13.47; 0, 24.30; S, 6.49).

### Example 31

### Synthesis of Rhodamine Peg Conjugates

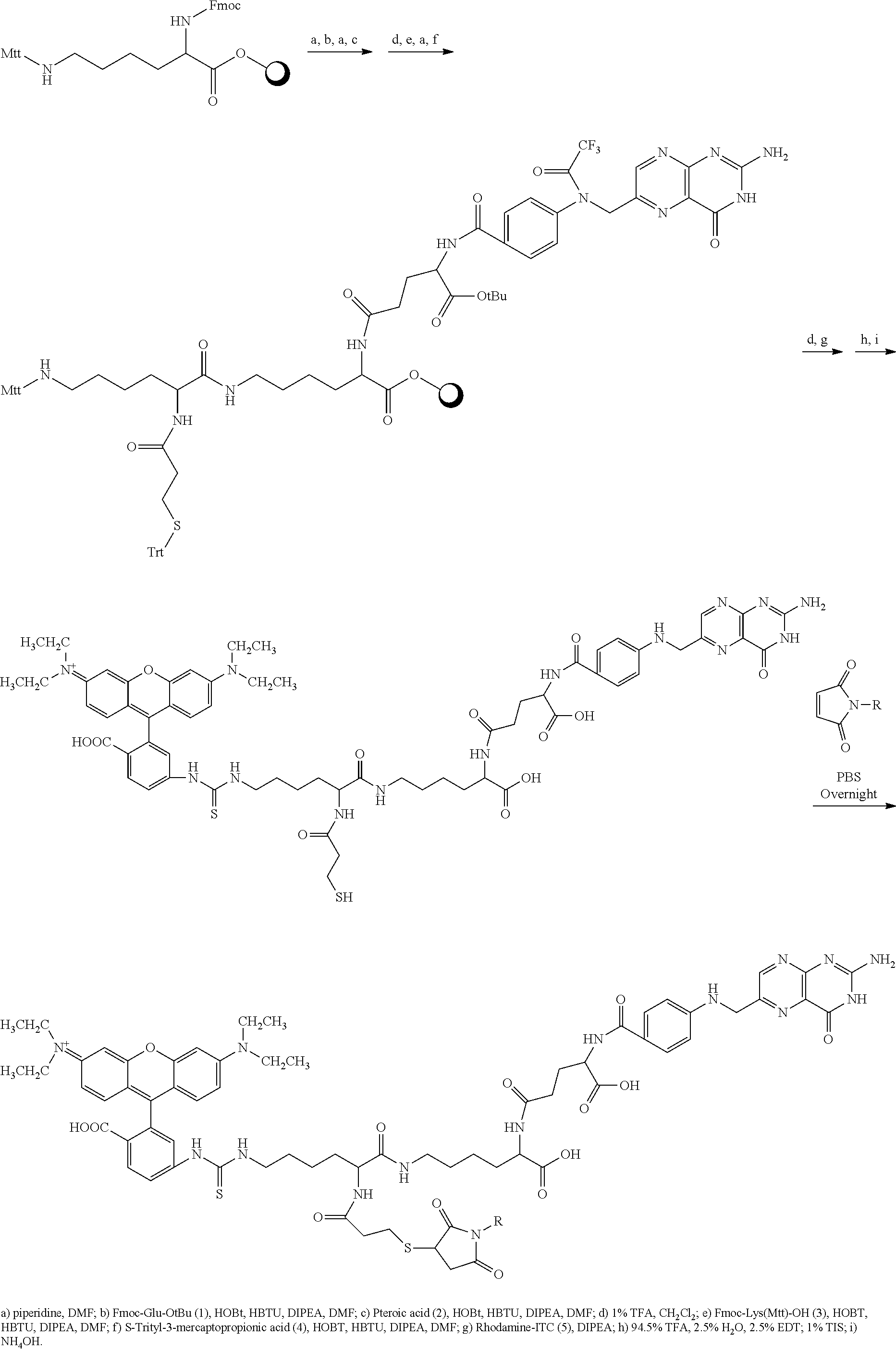

In the preceding scheme, R represents the following:

| | |
|---|---|
| mPEG(5k) | —(CH₂CH₂O)₇₆—CH₃ |
| mPEG(20k) | —(CH₂CH₂O)₄₅₄—CH₃ |
| mPEG2(60k) | 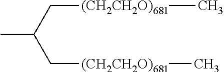 |

### Synthesis of Folate-Rhodamine-SH as a PEG-Anchor

Standard Fmoc peptide chemistry was used to synthesize a folate linked to Rhodamine B-isothiocyanate via a spacer composed of two lysines attached to the γ-COOH terminal of folic acid. The sequence Lys-Lys-(γ)Glu-pteroic acid was constructed by Fmoc chemistry with HBTU and HOBT (Novabiochem, San Diego, Calif.) as the activating agents along with diisopropyethylamine (DIPEA) as the base. The Fmoc groups were deprotected with 20% piperidine in dimethylformamide (DMF). α-Fmoc-protected lysine-loaded Wang resin, containing a 4-methyltrityl protecting group on the ε-amine, was used as an anchor for folate. An Fmoc-Glu-OtBu was linked to the α-amine of the lysine to provide a γ-linked conjugate of folate after N¹⁰-trifluoroacetylpteroic acid (SIGMA, St. Louis, Mo.) was attached to the glutamic acid amine. The methoxytrityl (Mtt) protecting group on the e-amine of lysine was removed with 1% trifluoroacetic acid in dichloromethane to allow attachment of a second Fmoc-Lys(Mtt)-OH. After removing the Mtt-protecting group of the second lysine, S-Trityl-protected 3-mercaptopropionic acid was coupled to the α-amine of the second lysine, using the coupling reagents, HOBT and HBTU as described above. Finally, the Mtt-protecting group of the second lysine was removed and Rhodamine-B isothiocyanate (SIGMA, St. Louis, Mo.) dissolved in DMF was reacted overnight with the peptide in the presence of DIPEA, and then washed thoroughly from the peptide resin beads. The resin was washed several times with dichloromethane, and methanol and left to dry under N₂ for several hours. The folate-Lys-Lys-mercaptopropionic acid-rhodamine peptide was then cleaved from the resin with 95% TFA/2.5 H₂O/2.5% TIS/2.5% EDT solution for 3-4 hours. Ice cold diethyl ether was used to precipitate the product, and the precipitant was collected by centrifugation. The product was then washed three times with diethyl ether and dried under vacuum. To remove the N¹⁰-trifluoracetyl protecting group from the folate moiety, the product was dissolved in 10% ammonium hydroxide solution and stirred for 30 min at room temperature under argon to prevent disulfide bonds from forming. The product was then lyophilized until dry and stored under argon. The product was confirmed by mass spectroscopic analysis ([M⁻] calculated, 1286.5. found, 1285.08).

Synthesis of Folate-PEG(5k)-Rhodamine, Folate-PEG(20k)-Rhodamine, and Folate-PEG(60k)-Rhodamine

The folate-rhodamine-SH anchor, synthesized as described above, was used to react with maleimide-activated PEG(5k), PEG(20k), or PEG(60k) (Nektar Therapeutics, San Carlos, Calif.). The PEG-MAL molecules were dissolved in PBS and a 5-fold molar excess of folate-rhodamine-SH was added to the solution and stirred overnight, at room temperature, under nitrogen. The non-reacted folate-rhodamine was then separated from the folate-PEG-rhodamine conjugate by gel filtration chromatography, using a coarse Sephadex G-50 column equilibrated in water, (fractionation range for globular proteins: 1,500-30,000, SIGMA, St. Louis, Mo.), and using gravity for running the samples. The folate-PEG-rhodamine peak was collected, lyophilized and re-suspended in phosphate buffered saline (PBS) for animal studies.

### Characterization of the Molecular Weight of Folate-PEG-Rhodamine Conjugates

In order to characterize the apparent molecular weight of the folate-PEG-rhodamine conjugates, their Vₑ/Vₒ ratio was compared with the Vₑ/Vₒ of protein standards of known molecular weight (Vₑ is the elution volume, and Vₒ is the void volume). Columns were run in phosphate buffered saline (PBS, pH 7.4), at room temperature, at a flow rate of 5 ml/min. The void volume of the column (Vₒ) was determined spectrophotometrically by the elution volume for blue dextran (molecular weight approx. 2,000,000, SIGMA, St. Louis, Mo.) at 610 nm, by measuring the volume of effluent collected from the point of sample application to the center of the effluent peak. Individual protein standards were dissolved in the PBS and their elution time was followed by absorbance readings at 280 nm. The elution volume (Vₑ) of the protein standards was determined by measuring the volume of effluent collected from the point of sample application to the center of the effluent peak. In order to determine the Vₑ of the folate-PEG-rhodamine conjugates, samples were applied on the column and ran at the same flow rate as used for blue dextran and the protein standards. The Vₑ of the folate-PEG-rhodamine conjugates was determined using the same method applied to the standards. Plotting the logarithms of the known molecular weights of protein standards versus their respective Vₑ/Vₒ values produces a linear calibration curve. Two different Sephacryl HR columns were used for the purpose of resolving all the folate-PEG-conjugates. A 24 cm×1.0 cm Sephacryl 100-HR (MW range 1000-10,000 Da) was able to resolve the folate-PEG(5k)-rhodamine conjugate, but not the folate-PEG(20k)-rhodamine and folate-PEG(60k)-rhodamine conjugates. The latter two conjugates were resolved on a 22 cm×1.0 cm Sephacryl 200-HR (MW range 5-250 kDa). The protein standards used on the Sephacryl 100-HR were: bradykinin fragment 2-9 (MW˜904), aprotinin from bovine lung (MW 6,511.44), myoglobin from horse heart (MW˜17,000), carbonic anhydrase from bovine erythrocytes (MW˜29,000), albumin (MW˜66,000), aldolase (MW˜161,000). The protein standards used on Sephacryl 200-HR were: myoglobin from horse heart (MW˜17,000), carbonic anhydrase from bovine erythrocytes (MW˜29,000), albumin (MW˜66,000), alcohol dehydrogenase from yeast (MW˜150,000), β-amylase from sweet potato (MW˜200,000), apoferritin from horse spleen (MW˜443,000), bovine thyroglobulin (MW˜669,000).

### Characterization of Folate/Rhodamine Ratio for Folate-PEG-Rhodamine Conjugates

In order to determine the ratio of folate to rhodamine on all the folate-PEG-rhodamine conjugates, first the extinction coefficients of folic acid and rhodamine-isothiocyanate in water were determined at two different wavelengths, 280 nm and 560 nm, by constructing standard curves at both these wavelengths. The slopes of these standard curves correspond to the extinction coefficients of folic acid and rhodamine-isothiocyanate in water. Samples of folate-PEG-rhodamine conjugates were then dissolved in water and their absorbances 280 nm and 560 nm were measured. The absorbances of folate-PEG-rhodamine conjugates at these wavelengths are due to both, the absorbance of folic acid (FA) and rhodamine (Rhod), therefore:

A₂₈₀=A₂₈₀(FA)+A₂₈₀(Rhod) and A₅₆₀=A₅₆₀(FA)+A₅₆₀(Rhod)

By using the extinction coefficients of folic acid (FA) and rhodamine (Rhod), determined by the standard curves, the concentrations of folate and rhodamine, and thus their ratio, in each folate-PEG-rhodamine conjugate sample can be determined by simultaneously solving for their respective concentrations in the following equations:

A₂₈₀=ε₂₈₀(FA)·1·c(FA)+ε₂₈₀(Rhod)·1·c(Rhod)

A₅₆₀=ε₅₆₀(FA)·1·c(FA)+ε₅₆₀(Rhod)·1·c(Rhod)

### Example 32

### Synthesis of Folate CW800

N¹⁰-TFA-Pteroic acid was synthesized as reported elsewhere. First, Fmoc-Lys(Mtt)-Wang resin was swelled in DMF for 20 min. The deprotection of Fmoc group on the resin was achieved by 20% piperidine in DMF. 2.5 e.q. Fmoc-(γ)Glu-OtBu, HOBT, HBTU and 4 e.q. DIPEA were added to the reaction. Two hours later, the Fmoc group on glutamic acid was deprotected with 20% piperidine. Then, 2.5 e.q. N¹⁰-TFA-Pteroic acid, HOBT and HBTU were dissolved in 3:1 DMF/DMSO and 4 e.q. DIPEA were added to the reaction and reacted for 4 h. The product was washed with DMF, DCM and methanol. 1% TFA/DCM was used to cleave the Mtt protection group. Cleavage of the conjugates was achieved by TFA:TIPS:H₂O (95:2.5:2.5). The crude product was then precipitated with cool ether. The crude product was then hydrolyzed with ammonium hydroxide (pH=10) for 20 min. Folate-lysine was purified by HPLC and characterized by MS and NMR. Folate-lysine and CW 800 succinimidyl ester (1:1) were stirred in 0.1 M carbonate buffer (pH 9.0) in the dark for 18 h. The folate-CW800 conjugates was purified by HPLC and characterized by MS and NMR.

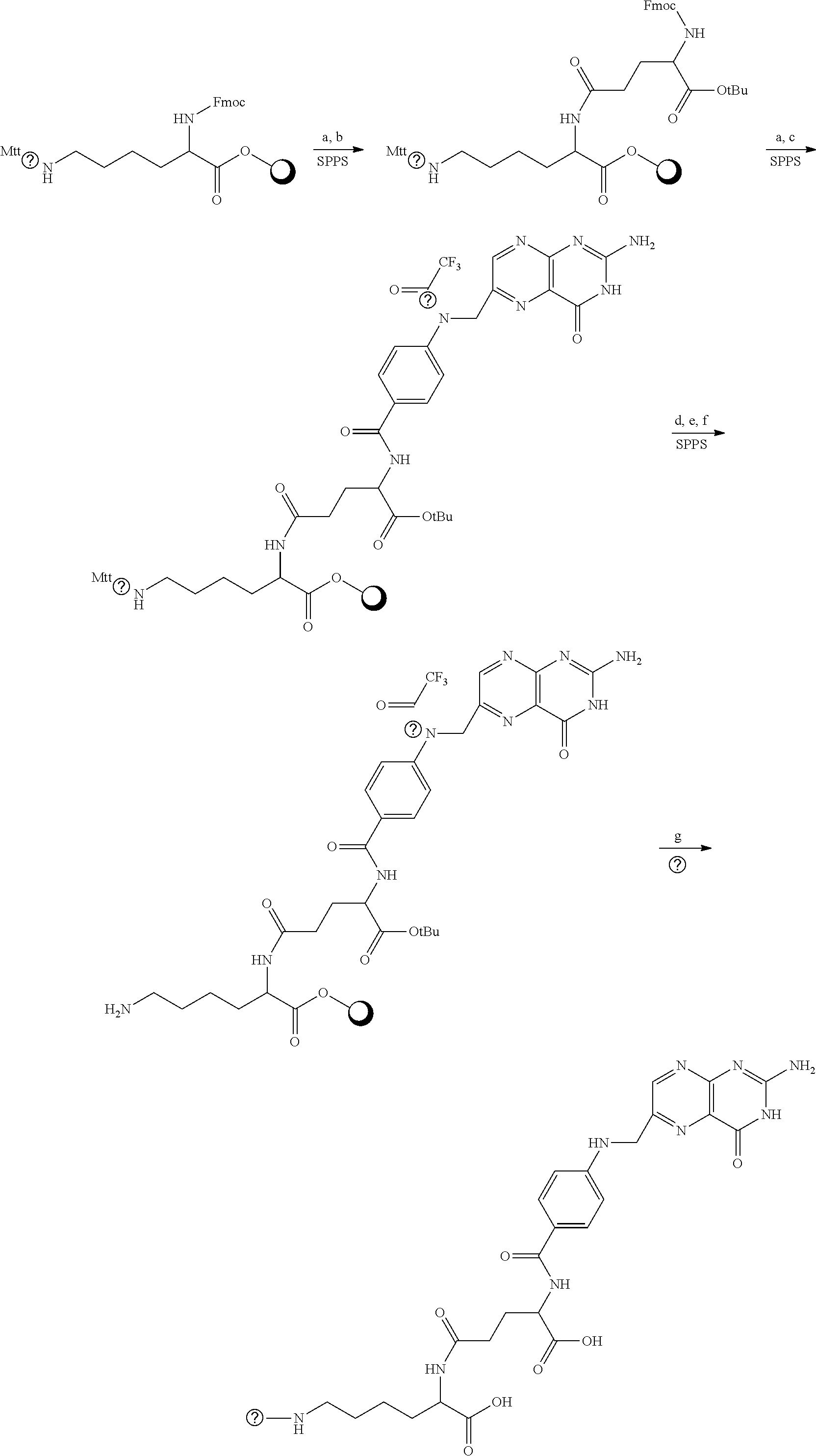

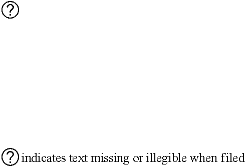

### Example 33

### Synthesis of Folate AlexaFluor 647

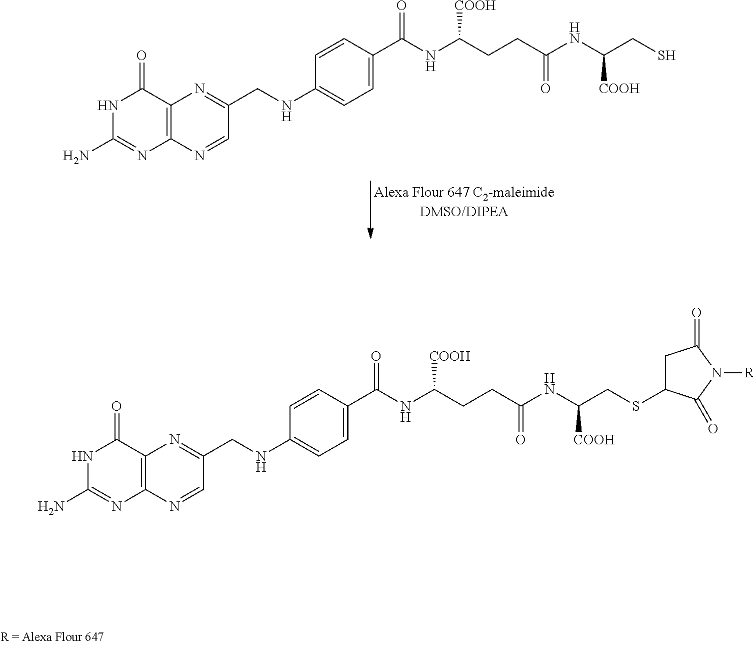

First, H-Cys(Trt)-2-Cl Trt resin was swelled in DMF for 20 min. The deprotection of Fmoc group on the resin was achieved with 20% piperidine in DMF. 2.5 e.q. Fmoc-(γ)Glu-OtBu, HOBT, HBTU and 4 e.q. DIPEA were added to the reaction. Two hours later, the Fmoc group on glutamic acid was deprotected with 20% piperidine. Then, 2.5 e.q. N¹⁰-TFA-Pteroic acid, HOBT and HBTU were dissolved in 3:1 DMF/DMSO and 4 e.q. DIPEA were added to the reaction and reacted for 4 h. The product was washed with DMF, DCM and methanol. Cleavage of the conjugates was achieved with TFA:TIPS:H₂O (95:2.5:2.5). The crude product was then precipitated with cool ether. The crude product was hydrolyzed with ammonium hydroxide (pH=10) for 20 min. Folate-cysteine was purified by HPLC and characterized by MS and NMR. Folate-cysteine and AlexaFluor 647 maleimide (Invitrogen, Carlsbad, Calif.; 1:1) were coupled in DMSO in the dark for 18 h. The folate-AlexaFluor 647 conjugate was purified by HPLC and characterized by MS and NMR.

It is to be understood that the foregoing Examples are merely illustrative of the compounds described herein and additional compounds may be prepared as described herein by the appropriate selection the starting materials, including the dye or fluorescent agent. For example, the following additional compounds are described.

### Example 34

### Synthesis of Folate-EDA-Rhodamine

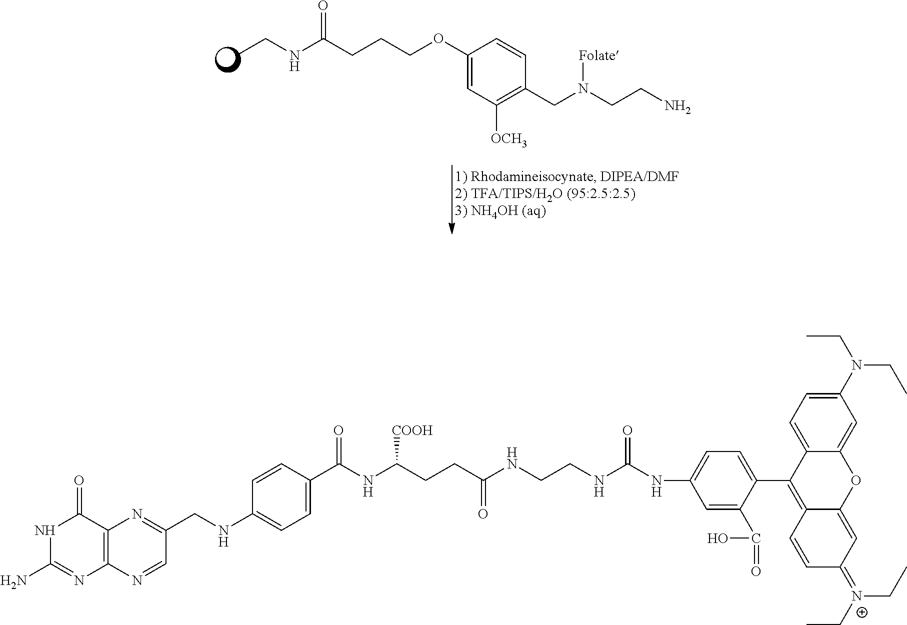

### Example 35

### Folate-EDA-Tetramethylrhodamine

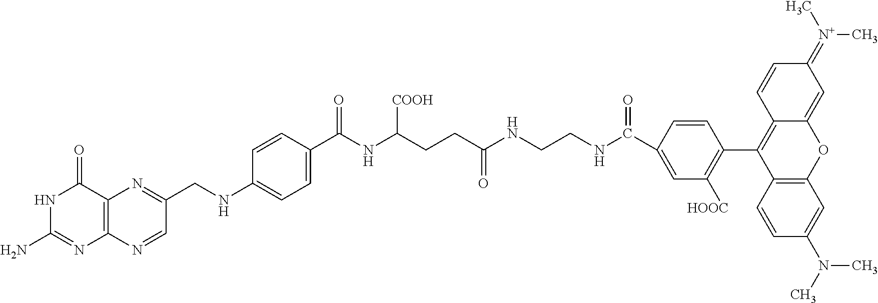 [SMILES: CN(C)c1ccc2c(-c3ccc(C(=O)NCCNC(=O)CCC(NC(=O)c4ccc(NCc5cnc6nc(N)[nH]c(=O)c6n5)cc4)C(=O)O)cc3C(=O)O)c3ccc(=[N+](C)C)cc-3oc2c1]

Folate-EDA-tetramethylrhodamine was prepared according to the process described above for Example 34.

### Example 36

### Synthesis of Folate-Lys-Rhodamine

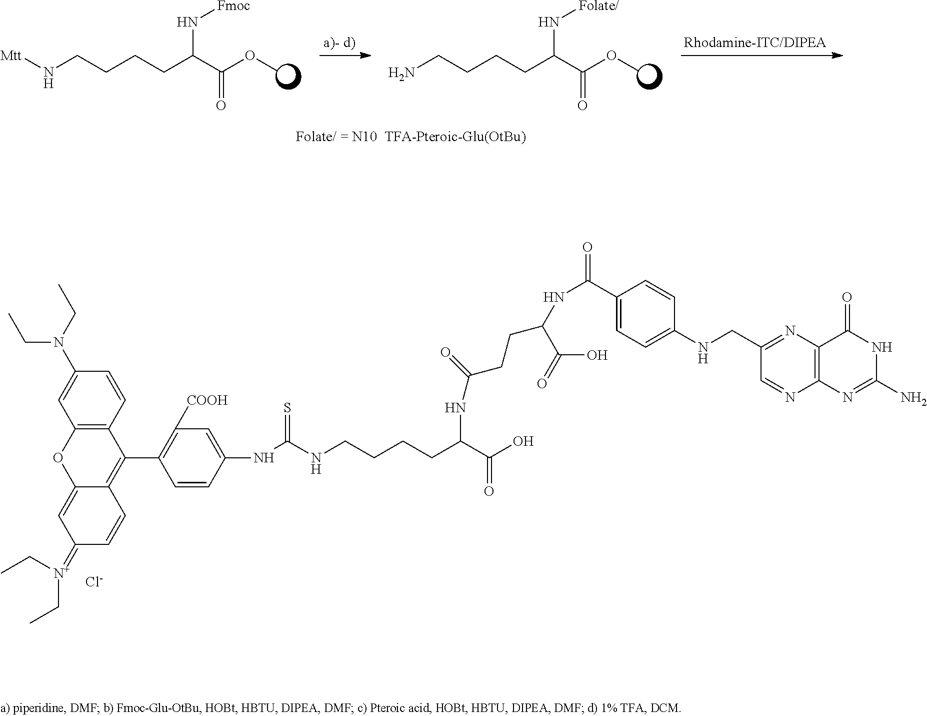

### Example 37

### Synthesis of Folate-AlexaFluor 488

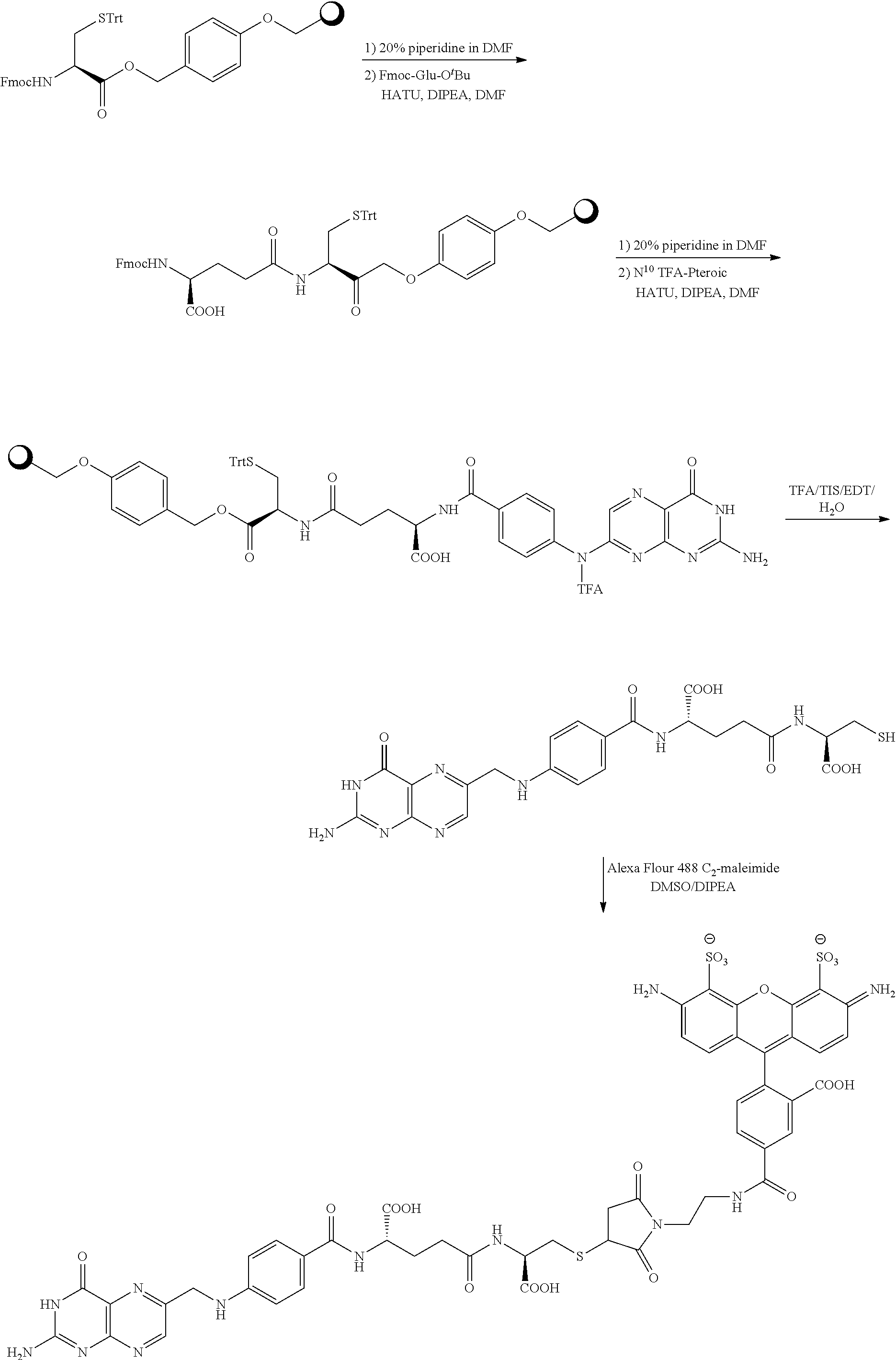

What is claimed is:

1. A method of detecting endothelial progenitor cells in a population of cells, said method comprising
contacting a population of cells with a composition comprising a conjugate or complex of the formula
A_{b}-X
where the group A_{b} comprises a folate that binds to endothelial progenitor cells and the group X comprises a fluorescent chromophore, and
detecting fluorescence from bound A_{b}-X, thereby detecting endothelial progenitor cells in a population of cells.

2. A method of detecting CD133⁺ Flk1⁺ endothelial progenitor cells in a population of progenitor cells, said method comprising
contacting a population of progenitor cells with a composition comprising a conjugate or complex of the formula
A_{b}-X
where the group A_{b} comprises a folate that binds to CD133⁺ Flk1⁺ endothelial progenitor cells and the group X comprises a fluorescent chromophore, and
detecting fluorescence from bound A_{b}-X, thereby CD133⁺ Flk1⁺ endothelial progenitor cells in a population of progenitor cells.

3. A method of quantifying CD133⁺ Flk1⁺ endothelial progenitor cells in a population of progenitor cells, said method comprising
contacting a population of progenitor cells with a composition comprising a conjugate or complex of the formula
A_{b}-X
where the group A_{b} comprises a folate that binds to CD133⁺ Flk1⁺ endothelial progenitor cells and the group X comprises a fluorescent chromophore,
detecting fluorescence from bound A_{b}-X, and
quantifying the percentage of fluorescing cells in said population, thereby quantifying CD133⁺ Flk1⁺ endothelial progenitor cells in a population of progenitor cells.

4. The method of claim 1, wherein the endothelial progenitor cells are CD133⁺ Flk1⁺ endothelial progenitor cells.

5. The method of claim 1, wherein the endothelial progenitor cells are common precursor cells.

6. The method of claim 1, wherein the fluorescent chromophore comprises a compound selected from fluorescein, Oregon Green, rhodamine, phycoerythrin, Texas Red, and AlexaFluor 488.

7. The method of claim 2, wherein the fluorescent chromophore comprises a compound selected from fluorescein, Oregon Green, rhodamine, phycoerythrin, Texas Red, and AlexaFluor 488.

8. The method of claim 3, wherein the fluorescent chromophore comprises a compound selected from fluorescein, Oregon Green, rhodamine, phycoerythrin, Texas Red, and AlexaFluor 488.

9. The method of claim 1, wherein A_{b}-X is folate-FITC.

10. The method of claim 2, wherein A_{b}-X is folate-FITC.

11. The method of claim 3, wherein A_{b}-X is folate-FITC.

12. The method of claim 1, wherein the population of cells is obtained from a human subject.

13. The method of claim 2, wherein the population of cells is obtained from a human subject.

14. The method of claim 3, wherein the population of cells is obtained from a human subject.

15. The method of claim 1, wherein the population of cells is obtained from a human subject suffering from a disease state mediated by the progenitor cells.

16. The method of claim 2, wherein the population of cells is obtained from a human subject suffering from a disease state mediated by the progenitor cells.

17. The method of claim 3, wherein the population of cells is obtained from a human subject suffering from a disease state mediated by the progenitor cells.

18. The method of claim 1, wherein the population of cells is obtained from a human subject suffering from a disease state selected from the group consisting of rheumatoid arthritis, osteoarthritis, ulcerative colitis, Crohn's disease, inflammatory lesions, infections of the skin, osteomyelitis, organ transplant rejection, pulmonary fibrosis, sarcoidosis, systemic sclerosis, lupus erythematosus, glomerulonephritis, restenosis, proliferative retinopathy, cancer, inflammations of the skin and any chronic inflammation.

19. The method of claim 2, wherein the population of cells is obtained from a human subject suffering from a disease state selected from the group consisting of rheumatoid arthritis, osteoarthritis, ulcerative colitis, Crohn's disease, inflammatory lesions, infections of the skin, osteomyelitis, organ transplant rejection, pulmonary fibrosis, sarcoidosis, systemic sclerosis, lupus erythematosus, glomerulonephritis, restenosis, proliferative retinopathy, cancer, inflammations of the skin and any chronic inflammation.

20. The method of claim 3, wherein the population of cells is obtained from a human subject suffering from a disease state selected from the group consisting of rheumatoid arthritis, osteoarthritis, ulcerative colitis, Crohn's disease, inflammatory lesions, infections of the skin, osteomyelitis, organ transplant rejection, pulmonary fibrosis, sarcoidosis, systemic sclerosis, lupus erythematosus, glomerulonephritis, restenosis, proliferative retinopathy, cancer, inflammations of the skin and any chronic inflammation.
